(19) United States
(12) Patent Application Publication
(10) Pub. No.: US 2023/0105604 A1
(43) Pub. Date: Apr. 6, 2023

(54) COMPOSITION INCLUDING I2SCN- IONS AND/OR I(SCN)2- IONS

(71) Applicant: TARADON LABORATORY, Tubize (BE)

(72) Inventors: Francoise BAFORT, Saint-Servais (BE); Mohamed Haïssam JIJAKLI, Scy (BE); Jean-Paul PERRAUDIN, Bruxelles (BE)

(73) Assignee: TARADON LABORATORY, Tubize (BE)

(21) Appl. No.: 17/747,572

(22) Filed: May 18, 2022

Related U.S. Application Data
(63) Continuation of application No. 16/897,706, filed on Jun. 10, 2020, now abandoned.
Application No. 16/897,706 is a continuation of application No. 15/761,211, filed on Apr. 13, 2018, now abandoned.

(30) Foreign Application Priority Data
Sep. 17, 2015 (FR) 1558780

(51) Int. Cl. C01C 3/00; A01N 59/24; A01N 59/12; C07K 14/475; C07K 14/79

(52) U.S. Cl. CPC C01C 3/005; A01N 59/24; A01N 59/12; C07K 14/475; C07K 14/79; C12Y 111/01007; C12Y 302/01017

(57) ABSTRACT

A stable composition obtained by enzymatic oxidation of a halide thiocyanate mixture, including at least one ion selected from the group of the I2SCN— ions and the I(SCN)₂— ions, the composition being free of hypothiocyanite ions. In an embodiment, the composition further includes iodine thiocyanate ISCN. This disclosure also relates to a method for preparing the stable composition and to the uses thereof.

This is a Continuation of application Ser. No. 16/897,706 filed Jun. 10, 2020, now abandoned, which in turn is a Continuation of application Ser. No. 15/761,211 filed Apr. 13, 2018, now abandoned, which claims the benefit of PCT Application No. PCT/EP2016/072088 filed Sep. 18, 2016, which claims the benefit of French Priority Application No. 1558780 filed Sep. 17, 2015. The disclosure of each of the prior applications is hereby incorporated by reference herein in its entirety.

The present invention relates to the production of antimicrobial compounds and to the uses thereof, including in combination with other molecules for the preparation of drugs, for the prophylaxis or the therapy of infectious diseases caused by microorganisms, for the protection of plants against their pathogenic agents and pests thereof, or also for the improvement of the quality of certain products such as certain paints, for example.

More precisely, it relates to the field of antimicrobial compounds produced by oxidation of thiocyanate ion in the presence of a halogen, said oxidation being catalyzed by means of particular enzymes called oxidoreductases, and more precisely peroxidases, the preferred enzyme being the lactoperoxidase (LP).

In the context of the present application, among the chemical elements of the 17^{th} column of the periodic table of elements (formerly referred to as group VII or VIIA), the term “halogen” denotes in particular chlorine (Cl), bromine (Br) and iodine (I).

The halogens can give rise to the formation of ions of “X⁻” type, referred to as “halide ions:” chloride ion (Cl⁻), bromide ion (Br⁻) and iodide ion (I⁻).

In general, the term “pseudo-halogen” denotes inorganic binary compounds of “MN” form, in which:

M is a cyanide (CN⁻), cyanate (OCN⁻) or thiocyanate (SCN⁻); and

N is one of these same groups or a true halogen as defined above.

In the context of the present application, the term “pseudo-halogen” will be understood to denote preferably the thiocyanate ions (SCN⁻).

In the context of the present application, the term “interhalogen” denotes compounds formed by several halogens as defined above, which may be identical or different. As an example, we can cite the triodide ion I₃⁻ (identical halogens) or also the iodine monochloride of formula ICI (different halogens).

In the context of the present application, the term “interpseudohalogen” denotes compounds formed by at least one halogen as defined above and thiocyanate (SCN⁻). As an example, we can cite the following species: I₂SCN⁻, ISCN and I(SCN)₂⁻.

In general, it is known that the halides (Cl⁻, Br⁻ or I⁻) or the pseudohalides (SCN⁻) can be oxidized, for example, in the presence of hydrogen peroxide (H₂O₂) (or of an H₂O₂ donor system).

The equations of the reactions are:

H₂O₂+X⁻(Cl⁻,Br⁻or I⁻)→OX⁻+H₂O

H₂O₂+SCN⁻→OSCN⁻+H₂O

These oxidation reactions can also be carried out in the presence of particular enzymes.

These oxidation reactions can also be carried out (with or without enzyme(s)) in the presence simultaneously of a halide ion (X⁻) and SCN⁻. These oxidation reactions can also be carried out in the presence of particular enzymes.

In biochemistry, the enzymes of the oxidoreductase class are notably classified into different groups: oxidases, reductases, peroxidases, oxygenases, hydrogenases, dehydrogenases, etc.

More particularly, the peroxidases are enzymes that are very widespread in the living world. In the organism, they decompose notably the toxic peroxide compounds.

In the laboratory, the peroxidases are very widely used, for example, notably horseradish peroxidase (HRP) is used extensively in biotechnology as a detection reagent in immunoassays.

In the group of the peroxidases, one distinguishes notably the heme perodidases.

The heme peroxidases are present in plants and in mammals.

Their role in plants is multiple: auxine metabolism, extracellular defense, biosynthesis and degradation of lignin, degradation of hydrogen peroxide, and oxidation of toxic reducing agents. The peroxidases of plants are induced by stress, for example, following an attack by pathogens, injuries, heat, cold, dryness or UV light.

As for the peroxidases of mammals, they play a role in the production of the thyroid hormone, in the detoxification of hydrogen peroxide, and also as a natural defense system against pathogens.

In the peroxidase group, one finds notably the lactoperoxidase (LP), the thyroid peroxidase (TPO), the myeloperoxidase (MPO), the salivary peroxidase (SPO) and the eosinophil peroxidase (EPO).

In the presence of specific substrates, the peroxidases will catalyze an oxidation reaction and generate oxidizing species which are responsible, for example, for the antimicrobial activity. The specificity of the substrates is characteristic of the type of peroxidase.

The lactoperoxidase (LP) is present in cow's milk at concentrations of approximately 30 mg/L, concentration which varies depending on the season, on the cow feed, but especially on the lactation stage (maximum concentration 3 to 5 days after calving).

One of its biological functions consists of a bacteriostatic/bactericidal effect in the presence of hydrogen peroxide (H₂O₂) and thiocyanate (SCN⁻). The lactoperoxidase (LP) can also oxidize certain halides, for example, the iodide ion (I).

The oxidation reactions catalyzed by lactoperoxidase (LP) can be summarized as follows:

H₂O₂+X⁻(Cl⁻,Br⁻or I⁻)+LP→OX⁻+H₂O+LP

H₂O₂+SCN⁻+LP→OSCN⁻+H₂O+LP

In the prior art, it is known that the species hypoiodite (OI⁻) and hypothiocyanite (OSCN⁻) have a bacteriostatic effect. These species will react, for example, with the groups NH₂ or thiols (—SH) of essential enzymes for the metabolism of the pathogen.

In the prior art, certain attempts to mix I⁻ and SCN⁻ ions before contact with hydrogen peroxide and the lactoperoxidase (LP) have been carried out.

The product sold under the name of KiB500®, which includes lactoperoxidase (LP) and thiocyanate ions, is known. This product is active against certain bacteria and viruses. The antimicrobial activity is due to the hypothiocyanite ions (OSCN).

In the publication Bosh et al. “The lactoperoxidase system: the influence of iodide and the chemical and antimicrobial stability over the period of about 18 months” (Journal of Applied Microbiology 2000, 89, 215-224), a system comprising the lactoperoxidase (LP), thiocyanate ions (SCN⁻) and iodide ions (I⁻) is studied regarding its bactericidal and fungicidal properties and their duration. From the diagrams on page 217, it can be deduced that the iodide ions improve the efficacy of the lactoperoxidase (LP) system. From FIGS. 4 and 5, it can be deduced that, when the system is stored in the presence of oxygen, the bactericidal activity decreases, notably when the composition is stored for more than 200 days. The authors specify that the ions formed are OI⁻ and OSCN⁻ ions which are known for their bactericidal/bacteriostatic activity. It is specified that these ions have a concentration that remains stable for approximately 1 month. The oxidation reactions take place “in parallel” between the SCN⁻ and I⁻ ions.

In the publication Bafort et al. “Mode of Action of Lactoperoxidase as Related to Its Antimicrobial Activity: A Review” (Hindawi Publishing Corporation Enzyme Research, Volume 2014, Article ID 517164, 13 pages), the active chemical species derived from thiocyanate ions or iodide ions by a lactoperoxidase (LP) system are studied. At the end of the article, it is specified that, with regard to the antimicrobial activity of a lactoperoxidase-iodide-thiocyanate system, contradictory results were obtained depending on the bacterial strain.

Thus, in the approaches of the prior art, when the lactoperoxidase (LP) system operates with SCN⁻ and I⁻, the latter compete for binding to the binding site of the lactoperoxidase (LP) in order to produce, in the presence of hydrogen peroxide (H₂O₂), jointly OSCN⁻ and OI⁻.

The reaction scheme described in the prior art is the following:

H₂O₂+SCN⁻+I⁻+lactoperoxidase (LP)→H₂O+OSCN⁻+Cl⁻+lactoperoxidase (LP).

Surprisingly, it has been observed that with the joint oxidation of I⁻ and SCN⁻ ions in the presence of H₂O₂ and in the presence, for a limited duration, of lactoperoxidase (LP) and at a pH from 4 to 8, the composition obtained has a greatly increased antimicrobial activity, that is to say greater than the compositions of the prior art comprising the OSCN⁻ and OI⁻ ions.

Under particular conditions, it has been demonstrated that species other than the OSCN⁻ and OI⁻ ions are formed, and even more surprisingly it has been shown that there is nor even any formation of OSCN⁻ ions.

It has been demonstrated that the species thus formed are not only highly active but moreover much more stable than the traditional compositions comprising OSCN⁻ and OI⁻ ions.

The solution obtained is free of OSCN⁻ ions and includes a mixture of the following ions of the “interpseudohalogen” type: I₂SCN⁻ and I(SCN)₂⁻.

In the prior art, these two species I₂SCN⁻ and I(SCN)₂⁻ were cited by Lewis, C. & Skoog, D. A., 1962. Spectrophotometric study of a thiocyanate complex of iodine, Journal of the American Chemical Society, 84(7), pp. 1101-1106.

Compositions obtained by chemical oxidation of a halide/thiocyanate mixture have been described in the prior art notably in Lewis, C. & Skoog, D. A., 1962. Spectrophotometric study of a thiocyanate complex of iodine, Journal of the American Chemical Society, 84(7), pp. 1101-1106 and in WO2016/026946. Due to the extremely slow kinetics of formation, these compositions do nor make it possible to obtain compositions which include, as predominant chemical species originating from the oxidation of a halide thiocyanate mixture, an ion selected from the group consisting of the I₂SCN⁻ and/or I(SCN)₂⁻ ions, as demonstrated by the mass spectrum appended to WO2016/026946. Thus, as demonstrated in the examples, the antimicrobial activity of the compositions obtained is not comparable to that of the compositions according to the invention.

Compositions obtained by enzymatic oxidation of a halide/thiocyanate mixture have been described in the prior art, notably in EP1349457 or WO2016026946. These compositions include an enzyme which remains permanently in the composition. As demonstrated in the examples, the concomitant presence of the enzyme and of the I₂SCN⁻ and/or I(SCN)₂⁻ ions for a time exceeding 60 minutes degrades I₂SCN⁻ and/or I(SCN)₂⁻ and causes the complete disappearance of the I₂SCN⁻ and/or I(SCN)₂⁻ ions after 48 h of joint presence.

The invention relates to a stable composition comprising at least one ion selected from the group consisting of I₂SCN⁻ ions and ions I(SCN)₂⁻ ions, said composition being free of hypothiocyanite ions (OSCN⁻).

Said composition is obtained by enzymatic oxidation of a halide thiocyanate mixture.

In said composition, the predominant chemical species originating from the oxidation of a halide thiocyanate mixture is an ion selected from the group consisting of the I₂SCN⁻ and/or I(SCN)₂⁻ ions.

In an embodiment, the stable composition according to the invention is characterized in that it includes I₂SCN⁻ and I(SCN)₂⁻ ions in combination, said composition being free of hypothiocyanite ions (OSCN⁻).

In an embodiment, the stable composition according to the invention is characterized in that it includes the I₂SCN⁻ ion.

In an embodiment, the stable composition according to the invention is characterized in that it includes the I(SCN)₂⁻ ion.

In an embodiment, the stable composition according to the invention is characterized in that it further comprises iodine thiocyanate ISCN.

In an embodiment, the stable composition according to the invention is characterized in that it further comprises at least one compound selected from the group consisting of lactoferrin, lysozyme, immunoglobulins, growth factors and mixtures thereof.

In an embodiment, the stable composition according to the invention is characterized in that it further comprises at least one compound selected from the group consisting of lactoferrin, lysozyme, immunoglobulins, one or more growth factor(s) and mixtures thereof.

In an embodiment, the stable composition according to the invention is characterized in that it further comprises at least one compound selected from the group consisting of lactoferrin, lysozyme, immunoglobulins, one or more growth factor(s) and mixtures thereof, characterized in that at least one growth factor is selected from the group consisting of Platelet Derived Growth Factor (PDGF), Fibroblast Growth Factor (FGF), Transforming Growth Factor (TGF), angiogenin, Epidermal Growth Factor (EGF), or a mixture thereof.

In an embodiment, the stable composition according to the invention is characterized in that it further comprises at least one compound selected from the group consisting of lactoferrin, lysozyme, immunoglobulins, one or more growth factor(s) and mixtures thereof, characterized in that at least one growth factor is supplied by a nutrient source, said nutrient source being skimmed or non-skimmed whey colostrum, or skimmed or non-skimmed colostrum.

In an embodiment, the stable composition according to the invention is characterized in that it further comprises at least lactoferrin.

In an embodiment, the stable composition according to the invention is characterized in that it further comprises at least lysozyme.

In an embodiment, the stable composition according to the invention is characterized in that it further comprises at least one immunoglobulin.

In an embodiment, the stable composition according to the invention is characterized in that it further comprises at least one growth factor.

In an embodiment, the stable composition according to the invention is characterized in that it further comprises at least one compound selected from the group consisting of lactoferrin, lysozyme, immunoglobulins, as well as at least one growth factor.

In an embodiment, the stable composition according to the invention is characterized in that it further comprises at least one compound selected from the group consisting of oils, spreading agents, emulsifiers, lubricants, adhesives and mixtures thereof.

In an embodiment, the stable composition according to the invention is characterized in that it further comprises at least one compound selected from the group consisting of sodium lauryl sulfate, magnesium stearate, lecithin, ethoxylated alcohols, plant oils, mineral oils, animal oils, polyoxyethylene sorbitol hexaoleate, carboxymethylcellulose (CMC), xanthan gums, gums arabic, starch and mixtures thereof.

In an embodiment, the stable composition according to the invention is characterized in that it further comprises at least one compound selected from the group consisting of sodium lauryl sulfate, magnesium stearate, lecithin, ethoxylated alcohols, plant oils, phosphatidyl plant oils, mineral oils, animal oils, polyoxyethylene sorbitol hexaoleate, carboxymethylcellulose (CMC), xanthan gums, gums arabic, starch and mixtures thereof, which enables it to strengthen, for example, the stability of the adhesion in certain applications.

The invention also relates to a stable composition comprising chemical entities comprising iodine atoms, in which the entities which are present in the largest number and which include at least one iodine atom are selected from the group consisting of the I₂SCN⁻, I(SCN)₂⁻ ions and mixtures thereof.

The invention also relates to a stable composition comprising chemical entities comprising iodine atoms, in which at least 50% of the iodine atoms of said compositions are involved in ions selected from the group consisting of the ions I₂SCN⁻, I(SCN)₂⁻ and mixtures thereof.

The invention also relates to a method for manufacturing a composition according to the invention, comprising:
a step A of preparation of a reaction medium comprising at least two substrates, at least one oxidizing agent, and a catalyst, the bringing together of said catalyst and said oxidizing agent being contingent upon the bringing together of said two substrates;
a reaction step B starting with the bringing together of said oxidizing agent and said catalyst;
a step C of removal of said catalyst, and of recovery of a composition according to the invention comprising at least I₂SCN⁻ ions and/or I(SCN)₂⁻ ions;
said substrates being halide (X—) and thiocyanate (SCN—) ions, said oxidizing agent being a system generating hydrogen peroxide (H₂O₂) and/or hydrogen peroxide, the catalyst being at least one peroxidase, said method being characterized in that said reaction step has a duration from 30 to 1800 seconds and in that it does nor give rise to the formation of hypothiocyanite ion (OSCN⁻).

In an embodiment, said bringing together of said substrates is simultaneous.

In an embodiment said bringing together of said substrates is sequential.

In an embodiment, the halide ions are iodide ions.

In an embodiment, the method according to the invention is characterized in that the composition according to the invention recovered at the end of step C includes I₂SCN⁻ ions and/or I(SCN)₂⁻ ions.

In an embodiment, the method according to the invention is characterized in that the reaction medium is an aqueous solution.

In an embodiment, the method according to the invention is characterized in that said halide (X⁻) and thiocyanate (SCN⁻) ions are added to said reaction medium in a powder form or in the form of a solution.

In an embodiment, the method according to the invention is characterized in that said halide (X⁻) and thiocyanate (SCN⁻) ions are added to said reaction medium in a powder form.

In an embodiment, the method according to the invention is characterized in that said halide (X⁻) and thiocyanate (SCN⁻) ions are added to said reaction medium in the form of a solution.

In an embodiment, the method according to the invention is characterized in that said halide (X⁻) and thiocyanate (SCN⁻) ions are added to said reaction medium in the form of a powder and a solution respectively, or in the form of a solution and a powder, respectively.

In an embodiment, the method according to the invention is characterized in that said medium obtained at the end of step C is a composition according to the invention which is stable.

In a particular embodiment, the method for manufacturing an antimicrobial composition includes at least the following steps:
Step 1: preparation of a solution A1 comprising at least one halide ion (X⁻);
Step 2: preparation of a solution A2 comprising at least one thiocyanate ion (SCN⁻);
Step 3: preparation of a component B consisting of a hydrogen peroxide (H₂O₂) generating system or of hydrogen peroxide in an aqueous solution;
Step 4: dipping of the peroxidase in an aqueous or buffered solution;
Step 5: contacting of the component A1 and A2 in the solution which contains the peroxidase;
Step 6: contacting of the compound B in the solution obtained in step 5 and maintaining of the contact for a time from 30 to 1800 seconds;
Step 7: removal of the peroxidase, and recovery of a composition according to the invention comprising at least X₂SCN⁻ ions and/or X(SCN)₂⁻ ions,
   wherein that said method does not give rise to the formation of hypothiocyanite ion (OSCN⁻).

Steps 1, 2, 3, 4 and 5 can be carried out in any order or simultaneously.

In an embodiment, the halide ions are iodide ions.

In a particular embodiment, the method for manufacturing an antimicrobial composition includes at least the following steps:
Step 1: preparation of a solution A1 comprising at least one iodide ion (I⁻);
Step 2: preparation of a solution A2 comprising at least one thiocyanate ion (SCN⁻);
Step 3: preparation of a compound B consisting of a hydrogen peroxide (H₂O₂) generating system or of hydrogen peroxide in an aqueous solution;
Step 4: dipping of the peroxidase in an aqueous or buffered solution;
Step 5: contacting of the compound A1 and A2 in the solution which contains the peroxidase;
Step 6: contacting of the component B in the solution obtained in step 5 and maintaining of the contact for a time from 30 to 1800 seconds;
Step 7: removal of the peroxidase, and recovery of a composition according to the invention comprising at least I₂SCN⁻ ions and/or I(SCN)₂⁻ ions,
   wherein said method does not give rise to the formation of hypothiocyanite ion (OSCN⁻).

Steps 1, 2, 3, 4 and 5 can be carried out in any order or simultaneously.

At the end of the method, the composition according to the invention can be subjected to a step of lyophilization at the end of which a lyophilisate is obtained, which, during a redissolution, makes it possible to reconstitute an antimicrobial composition according to the invention which includes X₂SCN⁻ ions and/or X(SCN)₂⁻ ions and which is free of hypothiocyanite ion (OSCN⁻).

At the end of the method, the composition according to the invention can be subjected to a step of lyophilization at the end of which a lyophilisate is obtained, which, during a redissolution, makes it possible to reconstitute an antimicrobial composition according to the invention which includes ions I₂SCN⁻ ions and/or I(SCN)₂⁻ ions and which is free of hypothiocyanite ion (OSCN⁻).

At the end of the method, the composition according to the invention can be subjected to a step of lyophilization at the end of which a lyophilisate is obtained, said lyophilisate enabling, during a redissolution, the reconstitution of said composition which includes I₂SCN⁻ and I(SCN)₂⁻ ions in combination and which is free of hypothiocyanite ion (OSCN⁻).

The thiocyanate ion (SCN⁻) can be supplied in any form, for example, in the form of potassium thiocyanate (KSCN) or sodium thiocyanate (NaSCN).

The iodide ion (I⁻) can be supplied in any form, for example, in the form of potassium iodide (KI) or sodium iodide (NaI) or else in the form of diiodine (I₂). In an embodiment, the iodide ion (I⁻) is supplied in the form of potassium iodide (KI).

In an embodiment, the iodide ion (I⁻) is supplied in the form of diiodine (I₂).

The expressions “does not give rise to the formation of hypothiocyanite ion (OSCN⁻)” or “composition free of hypothiocyanite ion (OSCN⁻)” are understood to mean that no peak indicating the formation of hypothiocyanite ion (OSCN⁻) is observed in NMR and anionic chromatography during the analysis of the composition.

The antimicrobial composition obtained is stable; “stable” is understood to mean a composition which loses its properties only to a very slight extent over time. For example, it can be an antimicrobial composition which loses less than 20% of its activity in 5 months when it is stored in a closed container at 4° C. with protection from light.

In an embodiment, the method for manufacturing a stable composition according to the invention is characterized in that the halide ion (X⁻) is selected from the group consisting of the iodide ion (I⁻), the bromide ion (Br⁻) and the chloride ion (Cl⁻).

In an embodiment, the method for manufacturing a stable composition according to the invention is characterized in that the halide ion (X⁻) is the iodide ion (I⁻).

In an embodiment, the method for manufacturing a stable composition according to the invention is characterized in that the iodide ion (I⁻) is in the form of potassium iodide (KI) or sodium iodide (NaI) or diiodine.

In an embodiment, the method for manufacturing a stable composition according to the invention is characterized in that the iodide ion (I⁻) is in the form of potassium iodide (KI).

In an embodiment, the method for manufacturing a stable composition according to the invention is characterized in that the iodide ion (I⁻) is in the form of sodium iodide (NaI).

In an embodiment, the method for manufacturing a stable composition according to the invention is characterized in that the iodide ion (I⁻) is in the form of a diiodine.

In an embodiment, the method for manufacturing a stable composition according to the invention is characterized in that the halide ion (X⁻) is present at a molar concentration from 0.1 mM to 1 M.

In an embodiment, the method for manufacturing a stable composition according to the invention is characterized in that the halide ion (X⁻) is present at a molar concentration from 0.1 mM to 500 mM.

In an embodiment, the method for manufacturing a stable composition according to the invention is characterized in that the halide ion (X⁻) is present at a molar concentration from 0.1 mM to 100 mM.

In an embodiment, the method for manufacturing a stable composition according to the invention is characterized in that the halide ion (X⁻) is present at a molar concentration from 0.1 mM to 10 mM.

In an embodiment, the method for manufacturing a stable composition according to the invention is characterized in that the halide ion (X⁻) is the iodide ion (I⁻) and is present at a molar concentration from 0.1 mM to 10 mM.

In an embodiment, the method for manufacturing a stable composition according to the invention is characterized in that the thiocyanate ion (SCN⁻) is present at a molar concentration from 0.1 mM to 1 M.

In an embodiment, the method for manufacturing a stable composition according to the invention is characterized in that the thiocyanate ion (SCN⁻) is present at a molar concentration from 0.1 mM to 500 mM.

In an embodiment, the method for manufacturing a stable composition according to the invention is characterized in that the thiocyanate ion (SCN⁻) is present at a molar concentration from 0.1 mM to 100 mM.

In an embodiment, the method for manufacturing a stable composition according to the invention is characterized in that the thiocyanate ion (SCN⁻) is present at a molar concentration from 0.1 mM to 10 mM.

In an embodiment, the method for manufacturing a stable composition according to the invention is characterized in that the halide ion (X⁻) is the iodide ion (I⁻), the concentration of iodide ions (I⁻) is greater than the concentration of said thiocyanate ion (SCN⁻), and the pH of the solution is from 4 to 8.

In an embodiment, the method for manufacturing a stable composition according to the invention is characterized in that the ratio between the molar concentration of iodide ion (I⁻) and the molar concentration of thiocyanate (SCN⁻) is strictly greater than 1.

In an embodiment, the method for manufacturing a stable composition according to the invention is characterized in that the ratio between the molar concentration of iodide ion (I⁻) and the molar concentration of thiocyanate (SCN⁻) is from 1.5 to 40.

In an embodiment, the method for manufacturing a stable composition according to the invention is characterized in that the ratio between the molar concentration of iodide ion (I⁻) and the molar concentration of thiocyanate (SCN⁻) is from 1.5 to 20.

In an embodiment, the method for manufacturing a stable composition according to the invention is characterized in that the ratio between the molar concentration of iodide ion (I⁻) and the molar concentration of thiocyanate (SCN⁻) is from 1.5 to 20.

In an embodiment, the method for manufacturing a stable composition according to the invention is characterized in that the ratio between the molar concentration of iodide ion (I⁻) and the molar concentration of thiocyanate (SCN⁻) is from 4 to 5.

In an embodiment, the method for manufacturing a stable composition according to the invention is characterized in that the pH of the solution is from 4 to 8.

In an embodiment, the method for manufacturing a stable composition according to the invention is characterized in that the pH of the solution is from 4.4 to 7.5.

In an embodiment, the method for manufacturing a stable composition according to the invention is characterized in that said medium is buffered by a buffer selected from the group consisting of the citrate buffer, the phosphate buffer or the acetate buffer.

In an embodiment, the method for manufacturing a stable composition according to the invention is characterized in that said thiocyanate ion (SCN—) is in the form of potassium thiocyanate (KSCN) or sodium thiocyanate (NaSCN).

In an embodiment, the method for manufacturing a stable composition according to the invention is characterized in that said component B consists of a hydrogen peroxide (H₂O₂) generating system.

In an embodiment, the method for manufacturing a stable composition according to the invention is characterized in that said component B consists of a hydrogen peroxide (H₂O₂) generating system which is a glucose oxidase (GOD)/Glucose (Glc) system.

In an embodiment, the method for manufacturing a stable composition according to the invention is characterized in that the molar concentration of hydrogen peroxide is substantially equal to the sum of the concentrations of thiocyanate ion (SCN⁻) and of halide ion (X⁻).

In an embodiment, the method for manufacturing a stable composition according to the invention is characterized in that the molar concentration of hydrogen peroxide is substantially equal to the sum of the concentrations of thiocyanate ion (SCN⁻) and of halide ion (X⁻) which is iodide (I⁻).

In an embodiment, the method for manufacturing a stable composition according to the invention is characterized in that the time of contacting of the oxidizing agent and the catalyst in the presence of the substrates or the reaction time (step B or step 6) is from 30 to 1800 seconds;

In an embodiment, the method for manufacturing a stable composition according to the invention is characterized in that the time of contacting of the oxidizing agent and the catalyst in the presence of the substrates or the reaction time (step B or step 6) is from 30 to 900 seconds.

In an embodiment, the method for manufacturing a stable composition according to the invention is characterized in that the time of contacting of the oxidizing agent and the catalyst in the presence of the substrates or the reaction time (step B or step 6) is from 30 to 200 seconds.

In an embodiment, the method for manufacturing a stable composition according to the invention is characterized in that the time of contacting of the oxidizing agent and the catalyst in the presence of the substrates or the reaction time (step B or step 6) is from 30 to 100 seconds.

In an embodiment, the method for manufacturing a stable composition according to the invention is characterized in that the time of contacting of the oxidizing agent and the catalyst in the presence of the substrates or the reaction time (step B or step 6) is from 50 to 100 seconds.

In an embodiment, the method for manufacturing a stable composition according to the invention is characterized in that said removal of said peroxidase is carried out by means of a method selected from the group consisting of the use of a “teabag,” centrifugation, flocculation, contacting with a support to which the peroxidase is grafted, such as, for example, fibers, a textile, polymer resins, granules, etc.

In an embodiment, the method for manufacturing a stable composition according to the invention is characterized in that said removal of said peroxidase is carried out by means of the use of a “teabag.”

In an embodiment, the method for manufacturing a stable composition according to the invention is characterized in that the peroxidase is selected from the group consisting of the lactoperoxidase (LP), the thyroid peroxidase (TPO), the myeloperoxidase (MPO), the salivary peroxidase (SPO) and the eosinophil peroxidase (EPO).

In an embodiment, the method for manufacturing a stable composition according to the invention is characterized in that the peroxidase is the lactoperoxidase (LP).

The lactoperoxidase is a powder which one can mix, for example, with bentonite or immobilize in a liquid solution on beads made of cationic resin beads. These supports can be placed in a “teabag.”

For the immobilization of the LPO on cationic resin beads, certain beads fix +/−40 mg of LPO per mL of resin.

In an embodiment, the method for manufacturing a stable composition according to the invention is characterized in that said peroxidase has a concentration from 1 mg/L to 500 mg/L.

In an embodiment, the method for manufacturing a stable composition according to the invention is characterized in that said peroxidase has a concentration from 50 mg/L to 250 mg/L.

In an embodiment, the method for manufacturing a stable composition according to the invention is characterized in that it further comprises at least one step of immobilization of the composition according to the invention on an immobilizing substrate.

In the context of the present application, an “immobilizing substrate” is a material enabling the retention of said composition during handling, the latter being in liquid form or dry form (dry residue obtained after the evaporation of the composition according to the invention, or after lyophilization).

In an embodiment, the immobilizing substrate is a fibrous material.

In an embodiment, the immobilizing substrate is a fabric.

In an embodiment, the immobilizing substrate is an impregnated fabric.

During a redissolution or when applied on microorganisms, the immobilizing substrate makes it possible to reconstitute an antimicrobial composition according to the invention.

The invention also relates to an immobilizing substrate comprising a composition according to the invention.

The invention also relates to uses of the stable composition according to the invention for prophylactic and/or therapeutic purposes.

In an embodiment, the stable composition according to the invention is characterized in that it is used in the treatment of infections.

In an embodiment, the stable composition according to the invention is characterized in that it is used as an antibacterial agent.

In an embodiment, the stable composition according to the invention is characterized in that it is used as an antiviral agent.

In an embodiment, the stable composition according to the invention is characterized in that it is used as an antifungal agent.

In an embodiment, the stable composition according to the invention is characterized in that it is used for destroying a microorganism selected from the group consisting of Colletotrichum lindemuthanium, Fusarium avenaceurn, Septoria tritici, Verticillium dahliae, Phytophthora infestans, Pythium ultimum, Colletotrichum musae, Pencilhum italicum, Penicillium digitatum, Botrytis cinerea, Penicillium expansum, Pectobacterium atroseptica, Pseudomonas syringae pv syringae, Pectobacterium carotovorum, Erwynia amylovora, Pseudomonas syringae pv. tomato, Clavibacter michiganensis subsp. michiganensis, Kocuria rhizolia, Staphylococcus aureus, Enterobacter gergoviae, Escherichia coli, Klebsiella pneumoniae, Pseudomonas aeruginosa, Pseudomonas fluorescent, Pseudomonas putita, Aspergillus niger, Penicillium pinophilum, Candida albicans, Burkholderia cepacia, Pseudomonas aeruginosa, Staphylococcus aureus, Klebsiella oxytoca, Burkholderia multivorans, Achromobacter denitrificans, Pseudomonas aeruginosa, Stenotrophomonas maltophilia, Rhodococcus equi, Streptococcus equi, Streptococcus mutans and Streptococcus zooepidemicus.

In an embodiment, the stable composition according to the invention is characterized in that it is used for destroying a microorganism selected from the group consisting of Colletotrichum lindemuthanium, Fusarium avenaceum, Septoria tritici, Verticillium dahliae, Phytophthora infestans, Pythium ultimum, Colletotrichum musae, Pencillium italicum, Penicilliurn digitatum, Botrytis cinerea, Penicillium expansum, Pectobacterium carotovorum, Pseudomonas syringae pv syringae, Pectobacterium atroseptica, Erwynia amylovora, Pseudomonas syringae pv. tomato, Clavibacter michiganensis subsp. michiganensis, Kocuria rhizolia, Staphylococcus aureus, Enterobacter gergoviae, Escherichia coli, Klebsiella pneumonia, Pseudomonas aeruginosa, Pseudomonas fluorescent, Pseudomonas putita, Aspergillus niger, Penicillium pinophilum, and Candida albicans.

In an embodiment, the stable composition according to the invention is characterized in that it is used for destroying Xylella fastidiosa.

In an embodiment, the stable composition according to the invention is characterized in that it is used for destroying a microorganism selected from the group consisting of Veillonella alcalescens, Fusobacterium nucleatum, Actinomyces viscosus, Lactobacillus acidophilus, Streptococcus mutans, Porphyromonas gingivalis, Prevotella intermedia, Campylobacter species, Treponema socranskii species, Streptococcus species, Eikenella, Capnocytophaga species, and Selenomonas species.

In an embodiment, the stable composition according to the invention is characterized in that it is used for destroying a microorganism selected from the group consisting of Veillonella alcalescens, Fusobacterium nucleatum, Actinomyces viscosus, Lactobacillus acidophilus and Streptococcus mutans.

In an embodiment, the stable composition according to the invention is characterized in that it is used for destroying at least one microorganism selected from the group of the bacteria organized in the form of a biofilm.

In an embodiment, the stable composition according to the invention is characterized in that it is used for destroying at least one microorganism selected from the group of the bacteria organized in the form of a biofilm, responsible for parodontitis, selected from the group consisting of Veillonella alcalescens, Fusobacterium nucleatum, Actinomyces viscosus, Lactobacillus acidophilus and Streptococcus mutans.

In an embodiment, the stable composition according to the invention is characterized in that it is used for destroying at least one microorganism such as Candida albicans, organized in the form of a biofilm or in isolated form, which is responsible for glossitis, thrush, denture stomatitis, cheilitis, angular cheilitis, mycoses of the feet and of the nails.

In an embodiment, the stable composition according to the invention is characterized in that it has no effect on Streptococcus salivarius.

In an embodiment, the stable composition according to the invention is characterized in that it is used as a drug in humans and/or animals.

In an embodiment, the stable composition according to the invention is characterized in that it is used as a drug in humans.

In an embodiment, the stable composition according to the invention is characterized in that it is used as a drug in animals.

In an embodiment, the stable composition according to the invention is characterized in that it is used in the treatment and/or prevention of infections in humans and/or animals.

In an embodiment, the stable composition according to the invention is characterized in that it is used in the treatment of infections in humans.

In an embodiment, the stable composition according to the invention is characterized in that it is used in the treatment of infections in animals.

In an embodiment, the stable composition according to the invention is characterized in that it is used in the prevention of infections in humans.

In an embodiment, the stable composition according to the invention is characterized in that it is used in the prevention of infections in animals.

In an embodiment, the stable composition according to the invention is characterized in that it is used in the treatment of infections caused by at least one microorganism in humans and/or animals.

In an embodiment, the stable composition according to the invention is characterized in that it is used in the treatment of infections caused by at least one microorganism in horses.

In an embodiment, the stable composition according to the invention is characterized in that it is used in the treatment and/or prevention of infections caused by a microorganism forming a biofilm and/or in the so-called “planktonic” form.

In an embodiment, the stable composition according to the invention is characterized in that it is used in the treatment and/or prevention of infections caused by a microorganism forming a biofilm on the surface of human cells selected from the group consisting of skin cells, oral mucosal cells, cells from the otorhinolaryngological sphere, cells from the gastroenterological sphere, and cells from the urogenital sphere.

In an embodiment, the stable composition according to the invention is characterized in that it is used in the treatment and/or prevention of infections caused by microorganisms selected from the group consisting of bacteria, viruses, protozoa, yeasts, molds, fungi, parasites and the like.

In an embodiment, the stable composition according to the invention is characterized in that it is used in the treatment and/or prevention of infections caused by a microorganism which is a bacterium.

In an embodiment, the stable composition according to the invention is characterized in that it is used in the treatment and/or prevention of infections caused by a microorganism which is a bacterium selected from the group consisting of Shigella, Salmonella, E. coli, Vibreo colera, Pseudomonas (Ps. pyocyanea), Staphylococcus (Staph. albus, aureus), Streptococcus (Strep. viridans, Strep. faecalis, B Streptococcus), Proteus, Helicobacter pylori and the like, preferably H. pylori.

In an embodiment, the stable composition according to the invention is characterized in that it is used in the treatment and/or prevention of infections caused by a microorganism which is a virus.

In an embodiment, the stable composition according to the invention is characterized in that it is used in the treatment and/or prevention of infections caused by a microorganism which is an enveloped virus.

In an embodiment, the stable composition according to the invention is characterized in that it is used in the treatment and/or prevention of infections caused by a microorganism which is an enveloped virus selected from the group consisting of the herpes-causing viruses (preferably the paramyxoviruses of herpes simplex such as, for example, the parainfluenza viruses), the orthomyxoviruses (such as, for example, the influenza A and B virus), the rotaviruses, the coronaviruses, the herpes viruses (such as, for example, the VZV virus, the cytomegalovirus, the Epstein-Barr virus and the HHV6 virus) and the retroviruses (such as, for example, the human T lymphocyte leukemia virus 1, the bovine leukemia virus and the simian immunodeficiency virus (SIV)).

In an embodiment, the stable composition according to the invention is characterized in that it is used in the treatment of dental plaque, periodontal diseases, Helicobacter pylori ulcers, infections known under the name of “tourista,” bacterial vaginitis, vaginoses, cystitis, chlamydia infections, gastrointestinal infections, diarrhea, caries, gingivitis, mucositis, herpes, acne and molluscum contagiosum.

In an embodiment, the stable composition according to the invention is characterized in that it is used in the treatment of dental plaque, periodontal diseases, ulcers, infections known under the name of “tourista,” bacterial vaginitis, vaginoses, cystitis and chlamydia infections.

In an embodiment, the stable composition according to the invention is characterized in that it is used in the treatment of dental plaque and in that said microorganism present in the buccodental sphere is Candida albicans.

In an embodiment, the stable composition according to the invention is characterized in that it is used in the treatment of mucoviscidosis and that the microorganism present is selected from the group consisting of Burkholderia cepacia, Pseudomonas aeruginosa and Staphylococcus aureus.

In an embodiment, the stable composition according to the invention is characterized in that it is administered by the oral, topical or injectable route.

In an embodiment, the stable composition according to the invention is characterized in that it is administered by the oral route.

In an embodiment, the stable composition according to the invention is characterized in that it is administered by the topical route.

In an embodiment, the stable composition according to the invention is characterized in that it is administered by the injectable route.

In an embodiment, the stable composition according to the invention is characterized in that it is administered in the form of a gel, a mouth wash product, a toothpaste, tablets, soft gel capsules, pellets, powder, powder mixtures, an impregnated fabric, etc.

The compositions according to the invention can include, in addition to the above-mentioned compounds, any pharmaceutically acceptable excipient known to the person skilled in the art. Such materials should be nontoxic. The precise nature of the excipient can depend on a certain number of factors including the route of administration.

In a therapeutic context, i.e., when a therapeutic effect is desired, the dose administered corresponds to the “therapeutic dose,” which depends on several factors (route of administration, patient age, sex, etc.) known to the person skilled in the art, with it possible for the latter to determine said dose.

In an embodiment, the stable composition according to the invention is characterized in that it is used in the field of agriculture, horticulture, cultivation of plants intended for consumption, cultivation of plants intended to be displayed as ornamental plants, cultivation of fruit plants, the cultivation from bulbs, cultivation of potted plants, forest maintenance, the treatment of harvested fruits or seeds, the treatment of isolated roots, etc.

In an embodiment, the stable composition according to the invention is characterized in that it is used for destroying a microorganism in the treatment of plant pathologies caused by at least one phytopathogenic microorganism.

In an embodiment, the stable composition according to the invention is characterized in that it is used for destroying a phytopathogenic microorganism selected from the group consisting of the bacteria, the viruses and the fungi and the like.

In an embodiment, the stable composition according to the invention is characterized in that it is used for the treatment of plants contaminated by at least one phytopathogenic microorganism before or after the harvest.

In an embodiment, the stable composition according to the invention is characterized in that it is used for the treatment of plants contaminated by at least one phytopathogenic microorganism which is a bacterium or a virus.

In an embodiment, the stable composition according to the invention is characterized in that it is used for the treatment of plants contaminated by at least one phytopathogenic microorganism which is a bacterium.

In an embodiment, the stable composition according to the invention is characterized in that it is used for the treatment of plants contaminated by at least one phytopathogenic microorganism which is a bacterium selected from the group consisting of Erwinia chrysanthemi, Pseudomonas syringae, Xanthomonas camtestrise and Curtobactrium flaccumfaciens.

In an embodiment, the stable composition according to the invention is characterized in that it is used for the treatment of plants contaminated by at least one phytopathogenic microorganism which is selected from the group consisting of Erwinia amylovora, Pectobacterium carotovorum subsp. carotovorum, Pectobacterium atrosepticum, Pseudomonas syringae pv. syringae, Pseudomonas syringae pv. tomato and Clavibacter michiganensis subsp. michiganensis.

In an embodiment, the stable composition according to the invention is characterized in that it is used for the treatment of plants contaminated by at least one phytopathogenic microorganism which is a fungus.

In an embodiment, the stable composition according to the invention is characterized in that it is used for the treatment of plants contaminated by at least one phytopathogenic microorganism selected from the group consisting of Penicillium spp., Botryotinia spp. such as, for example, Botrytis cinerea, Didymella spp. such as, for example, Didymella lycopersici or Didymella bryonia, Pythium spp., Plasmopara spp., Peronospora spp., Sclerospora spp., Sphaerotheca spp. such as, for example, Sphaerotheca pannose and Sphaerotheca fulisinea, Puccunia spp. such as, for example, Puccunia horiana, Erysiphe spp., Oldium spp., Leveillula spp. such as, for example, Leveillula taurica, Fusarium spp., Phytophthora spp., Rhizoctonia spp., Verticillium spp., Sclerotinia spp., Rhizopus spp. and Ventura spp.

In an embodiment, the stable composition according to the invention is characterized in that it is used for the treatment of plants contaminated by at least one phytopathogenic microorganism selected from the group consisting of Colletotrichum lindemuthanium, Fusarium avenaceum, Septoria tritici, Verticillium dahlia, Phytophthora infestans, Pythium ultimum, Colletotrichum musae, Penicillium italicum, Penicillium digitatum, Botrytis cinerea and Penicillium expansum.

In an embodiment, the stable composition according to the invention is characterized in that it is used for the treatment of plants contaminated by at least one phytopathogenic microorganism selected from the group consisting of Colletotrichum lindemuthanium, Septoria tritici, Verticillium dahlia, Phytophthora infestans, Pythium ultimum, Colletotrichum musae, Penicillium italicum, Penicillium digitatum, Botrytis cinerea and Penicillium expansum.

In an embodiment, the stable composition according to the invention is characterized in that it is used for the treatment of plants contaminated by at least one phytopathogenic microorganism which is selected from the group consisting of Botrytis cinerea, Penicillium expansum, Penicillium italicum, Penicillium digitatum, Fusarium avenaceum, Phytophthora infestans, Verticillium dahlia, Colletotrichum lindemuthanium, Colletotrichum musae, Pythium ultimum, Venturia inaequalis, Plasmopara viticola, Erysiphe necator, Pectobacterium caratovorum, Pectobacterium atrosepticum, Clavibacter michiganensis subsp. michiganensis, Pseudomonas syringae pv. tomato, Pseudomonas syringae subsp. syringae, Erwinia amylovora, Xanthomonas orizae, and Xylella fastidiosa.

In an embodiment, the stable composition according to the invention is characterized in that it is used for the treatment of objects contaminated by at least one pathogenic microorganism selected from the group consisting of Kocuria rhizolia, Staphylococcus aureus, Enterobacter gergoviae, Escherichia coli, Klebsiella pneumoniae, Pseudomonas aeruginosa, Pseudomonas fluorescent, Pseudomonas putita, Aspergillus niger, Penicillium pinophilum, and Candida albicans.

In an embodiment, the stable composition according to the invention is characterized in that it is used in combination with another compound considered to combat at least one phytopathogenic microorganism in order to remedy the potential resistance problems.

In an embodiment, the stable composition according to the invention is characterized in that it is used in the treatment of plants according to a method selected from the group consisting of spraying, watering, atomization, aerial spraying, sprinkling, immersion, drip irrigation, bathing, etc.

In an embodiment, the stable composition according to the invention is characterized in that it is used for the disinfection of drip irrigation systems.

In an embodiment, the stable composition according to the invention is characterized in that it is used in a form selected from the group consisting of the liquid form and the solid form.

In an embodiment, the stable composition according to the invention is characterized in that it is in liquid form.

In an embodiment, the stable composition according to the invention is characterized in that it is in solid form.

### DESCRIPTION OF THE FIGURES

FIG. 1: NMR spectrum of a control composition without iodide

FIG. 1 is a ¹³C NMR spectrum of the control composition without iodide corresponding to test 2 of table 1 (conditions: 2.4 mM KSCN+2.4 mM H₂O₂+LP; phosphate buffer 100 mM pH 7.4). In this spectrum, several very clear peaks are observed: at 133.48 ppm, the peak corresponds to the thiocyanate ion (SCN⁻), and at 127.71 ppm, the peak corresponds to the hypothiocyanite ion (OSCN⁻). There is no peak in the vicinity of 50 ppm.

FIG. 2: NMR spectrum of a composition according to the invention

FIG. 2 is a ¹³C NMR spectrum of the composition corresponding to test 20 of table 1 (conditions: 5.4 mM KI+1.2 mM KSCN+6.6 mM H₂O₂+LP; phosphate buffer 100 mM pH 7.4). In this spectrum, the peak positioned at 50.36 ppm for this test, corresponding to the I₂SCN⁻ and I(SCN)₂⁻ ions, is observed. This peak is not observed in the control without KI (test 2, see FIG. 1). No peak corresponding to the thiocyanate ion (SCN⁻) or to the hypothiocyanite ion (OSCN⁻) can be observed.

FIGS. 3A-3C: NMR spectrum of a composition according to the invention

FIG. 3A is a ¹³C NMR spectrum of the composition corresponding to test 35 of table 2 (conditions: 5.4 mM KI+1.2 mM KSCN+6.6 mM H₂O₂+LP; sodium acetate buffer 100 mM pH 4.5). As specified above, the concentration of H₂O₂ is equal to the sum of the concentration of KI and the concentration of KSN. In this spectrum, the peak positioned at 49.6 ppm for this test, corresponding to the I₂SCN⁻ and I(SCN)₂⁻ ions, is observed. No peak corresponding to the thiocyanate ion (SCN⁻) or to the hypothiocyanite ion (OSCN⁻) can be observed.

FIG. 3B is a ¹³C NMR spectrum of the composition corresponding to test 33 of table 2 (conditions: 1.2 mM KI+1.2 mM KSCN+2.4 mM H₂O₂+LP; sodium acetate buffer 100 mM pH 4.4). As specified above, the concentration of H₂O₂ is equal to the sum of the concentration of KI and the concentration of KSN. In this spectrum, no peak is observed at 49.6 ppm for this test, corresponding to the I₂SCN⁻ and I(SCN)2⁻ ions. Peaks corresponding to other species, on the other hand, are observable.

FIG. 3C is a ¹³C NMR spectrum of the composition corresponding to test 30 of table 2 (conditions: 5.4 mM KI+0.6 mM KSCN+6.0 mM H₂O₂+LP; sodium acetate buffer 100 mM pH 4.4). As specified above, the concentration of H₂O₂ is equal to the sum of the concentration of KI and the concentration of KSN. In this spectrum, the peak positioned at 49.6 ppm for this test, corresponding to the I₂SCN⁻ and I(SCN)₂⁻ ions, is observed. No peak corresponding to the thiocyanate ion (SCN⁻) or to the hypothiocyanite ion (OSCN⁻) can be observed.

FIG. 4A: Mass spectrum of a composition according to the invention

FIG. 4a is a mass spectrum of a composition according to the invention prepared with ¹²C (conditions: 5.4 mM KI+5.4 mM H₂O₂+LP; ammonium acetate buffer 100 mM pH 4.5—concentrations of KSCN tested 1.2-2.4-4.8-5.4-7.2-10.6-0). The fragment at 311.78 corresponds to the I₂S¹²CN⁻ ion formed.

FIG. 4B: Mass spectrum of a composition according to the invention

FIG. 4b is a mass spectrum of a composition according to the invention prepared with ¹³C. (conditions: 5.4 mM KI+5.4 mM H₂O₂+LP; ammonium acetate buffer 100 mM pH 4.5—concentrations of KSCN tested 1.2-2.4-4.8-5.4-7.2-10.6). The fragment at 312.78 corresponds to the I₂S¹³CN⁻ ion formed.

FIG. 5: Variation of the survival rate of Candida albicans as a function of time

La FIG. 5 illustrates the variation of the survival rate of Candida albicans as a function of time, evaluated over a period of 6 months.

FIG. 6: Photographs of the culture media after contact versus control, after 5 minutes or 30 minutes

FIG. 6 consists of photographs of the culture media after contact versus control. Whether the contact time is 30 minutes (left portion of the figure) or 5 minutes (right portion of the figure), the visual result is the same: no colony of Candida albicans resists the composition according to the invention.

FIG. 7: Photographs of the culture media after contact versus control, at different concentrations

FIG. 7 consists of photographs of the culture media after contact versus control, at different concentrations. It is observed that even at a concentration of 25 μm, the number of colonies formed is greatly reduced in comparison to the control.

FIGS. 8A and 8B: Selectivity of a composition according to the invention

FIGS. 8A and 8B are photographs of the culture media after contact with Streptococcus mutans (cariogenic bacterium) FIG. 8A and Streptococcus salivarius (commensal bacterium) FIG. 8B: it can be deduced from this that the composition according to the invention has a bactericidal effect on Streptococcus mutans (FIG. 8A) and no bactericidal effect on Streptococcus salivarius (FIG. 8B).

FIG. 9: Activity on Xylella fastidiosa subsp. fastidiosa

FIG. 9 consists of photographs of the culture media after contact with Xylella fastidiosa subsp. fastidiosa: it can be deduced from this that the composition according to the invention has a bactericidal effect on Xylella fastidiosa subsp. fastidiosa (right portion of the figure), while the control has no effect (left portion of the figure).

FIG. 10: Activity on Xylella fastidiosa subsp. multiplex

FIG. 10 consists of photographs of the culture media after contact with Xylella fastidiosa subsp. multiplex: it can be deduced from this that the composition according to the invention has a bactericidal effect on Xylella fastidiosa subsp. multiplex (right portion of the figure), while the control has no effect (left portion of the figure).

FIG. 11: Activity on Xylella fastidiosa subsp. pauca.

FIG. 11 consists of photographs of the culture media after contact with Xylella fastidiosa subsp. pauca: it can be deduced from this that the composition according to the invention has a bactericidal effect on Xylella fastidiosa subsp. pauca (right portion of the figure), while the control has no effect (left portion of the figure).

FIG. 12: Absence of the I₂SCN⁻ and/or I(SCN)₂⁻ Ions in the prior art (Example 1; aqueous matrix)

Compositions obtained by enzymatic oxidation of a halide thiocyanate mixture have been described in the prior art, notably in EP1349457 or WO2016026946. Mixtures prepared in water according to the operating procedures described in these patent applications did not make it possible to obtain an ion selected from the group consisting of the I₂SCN⁻ and/or I(SCN)₂⁻ ions.

FIG. 13: Absence of the I₂SCN⁻ and/or I(SCN)₂⁻ Ions in the prior art (Example 2; acid buffered matrix)

Compositions obtained by enzymatic oxidation of a halide thiocyanate mixture have been described in the prior art, notably in EP1349457 or WO2016026946. Mixtures prepared in a citrate buffer 100 mM pH 5.5 according to the operating procedures described in these patent applications did not make it possible to obtain an ion selected from the group consisting of the I₂SCN⁻ and/or I(SCN)₂⁻ ions. For greater clarity, the signals corresponding to the carbons of the sodium citrate have been eliminated from the spectrum (see FIG. 14).

FIG. 14: NMR spectrum of sodium citrate and thiocyanate

The sodium citrate contains 4 carbons visible in NMR with the following chemical shifts: C₁ appears at 180.45 ppm, C₂ appears at 175.94 ppm, C₃ appears at 73.43 ppm and C₄ appears at 44.64 ppm. The thiocyanate contains 1 carbon, visible at the chemical shift of 133.48 ppm.

FIG. 15: Absence of the I₂SCN⁻ and/or I(SCN)₂⁻ ions in the prior art (Example 3; neutral buffered matrix)

Compositions obtained by enzymatic oxidation of a halide thiocyanate mixture have been described in the prior art, notably in EP1349457 or WO2016026946. Mixtures prepared in a phosphate buffer 100 mM pH 7.4 according to the operating procedures described in these patent applications did not make it possible to obtain an ion selected from the group consisting of the I₂SCN⁻ and/or I(SCN)₂⁻ ions.

FIG. 16: Importance of the matrix for the appearance of the I₂SCN⁻ and/or I(SCN)₂⁻ ions

Compositions obtained by enzymatic oxidation of a halide thiocyanate mixture in the ideal ratio of 4.5 (KI with respect to KSCN) in an aqueous matrix (of the slightly mineralized spring water, moderately mineralized spring water, highly mineralized spring water or tap water type), irrespective of the mineral composition of the aqueous matrix, did not make it possible to obtain an ion selected from the group consisting of the I₂SCN⁻ and/or I(SCN)₂⁻ ions.

FIG. 17: Presence of the OSCN⁻ ions in the prior art (Example 1; aqueous matrix)

Compositions obtained by enzymatic oxidation of a halide thiocyanate mixture have been described in the prior art, notably in EP1349457 or WO2016026946. Mixtures prepared in water according to the operating procedures described in these patent applications include hypothiocyanite ions.

FIG. 18: Presence of the OSCN⁻ Ions in the prior art (Example 2; acid buffered matrix)

Compositions obtained by enzymatic oxidation of a halide thiocyanate mixture have been described in the prior art, notably in EP1349457 or WO2016026946. Mixtures prepared in a citrate buffer 100 mM pH 5.5 according to the operating procedures described in these patent applications include hypothiocyanite ions.

FIG. 19: Enzymatic oxidation of the I₂SCN⁻ and/or I(SCN)₂⁻ Ions

The concomitant presence of the LP and of the I₂SCN⁻ and/or I(SCN)₂⁻ ions causes a degradation of the I₂SCN⁻ and/or I(SCN)₂⁻X ions with a decrease of the signal at 49 ppm after 1 h (slight decrease), 3 h of incubation (pronounced decrease of the signal), and total disappearance of the signal after 48 h.

FIG. 20: Synergy effect between the composition according to the invention and lactoferrin

FIG. 20 illustrates the synergy of antimicrobial activity with respect to P. expansum (10⁶ spores/mL) when the composition according to the invention is diluted 10-fold with an addition of lactoferrin (>2.5 mg/mL) compared to the same solution without addition of lactoferrin

FIG. 21: Action of the composition according to the invention on the biofilms

FIG. 21 illustrates the action which the composition according to the invention (“solution B;” points in the form of a triangle) has on the different bacteria organized in the form of a biofilm. It is apparent that, even with a reduced contact time (5 minutes), the population of the biofilm is very significantly reduced. This is not observed with the commercial composition of Chlorhexidine gluconate at 2% (“solution A;” points in the form of a square).

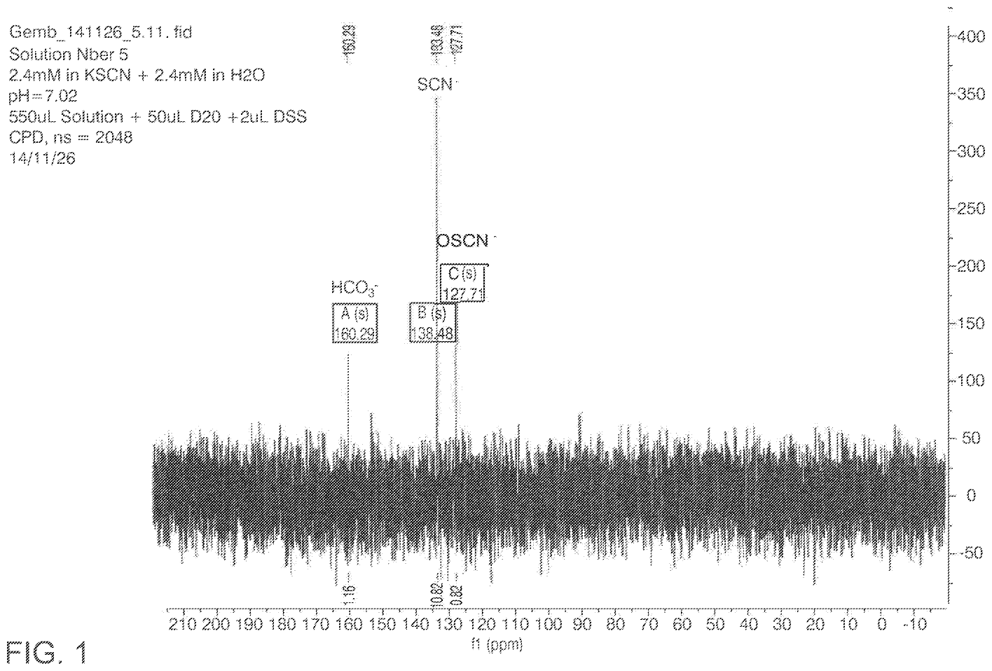

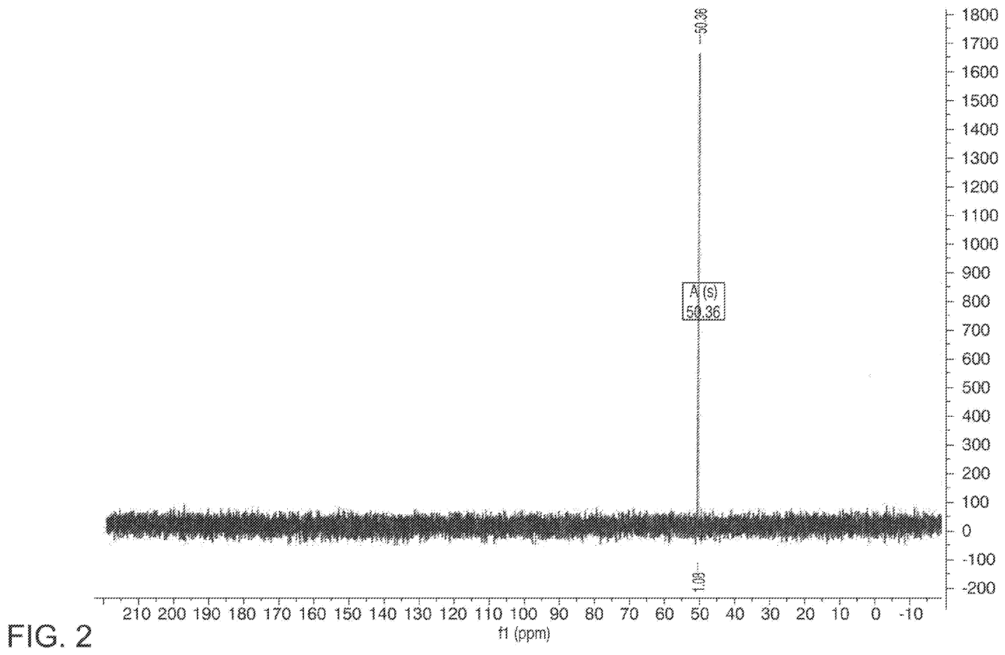

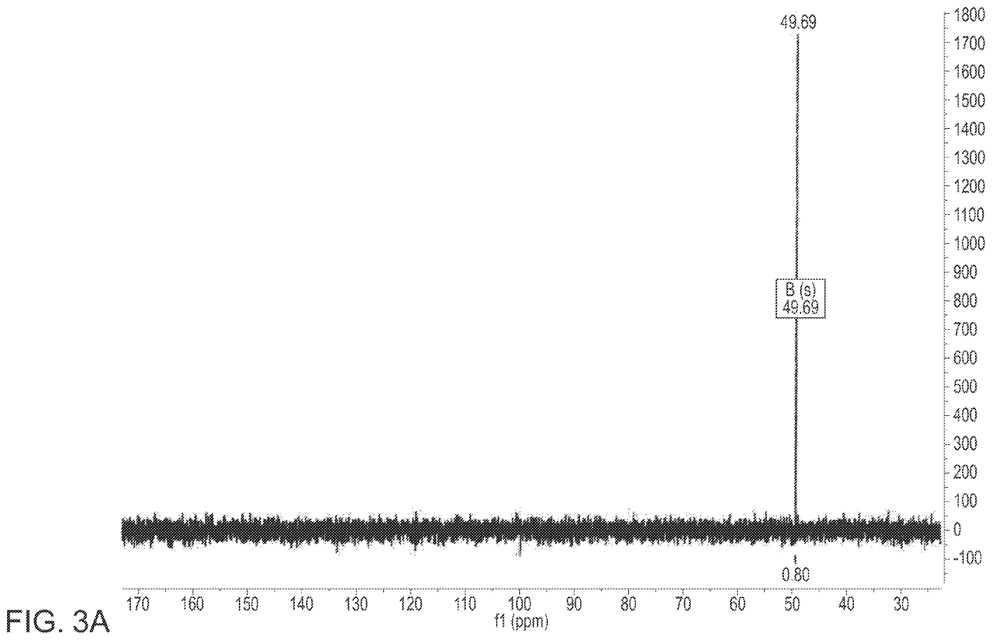

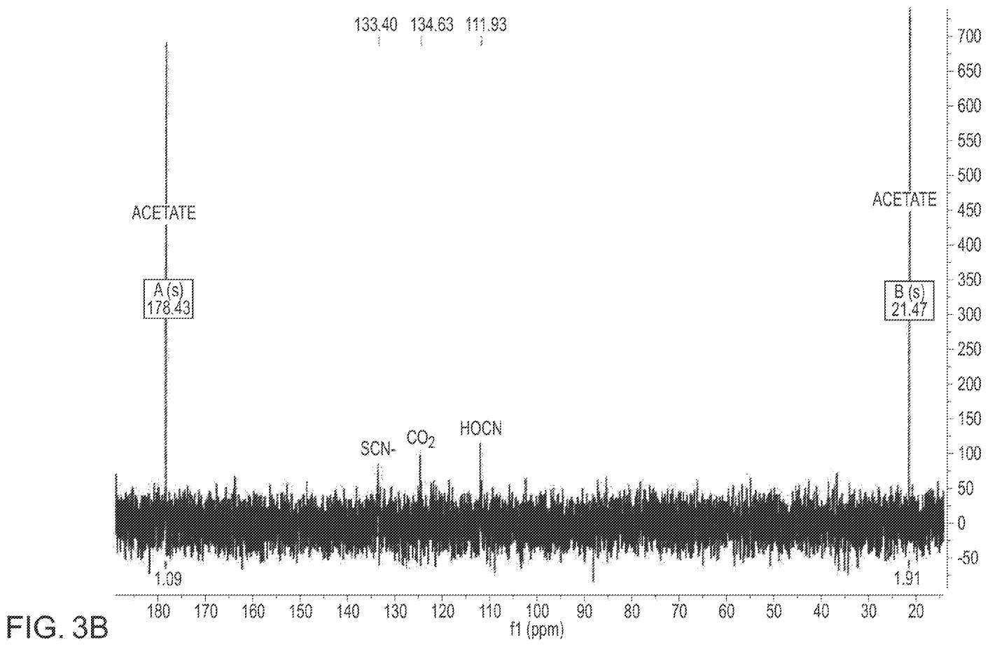

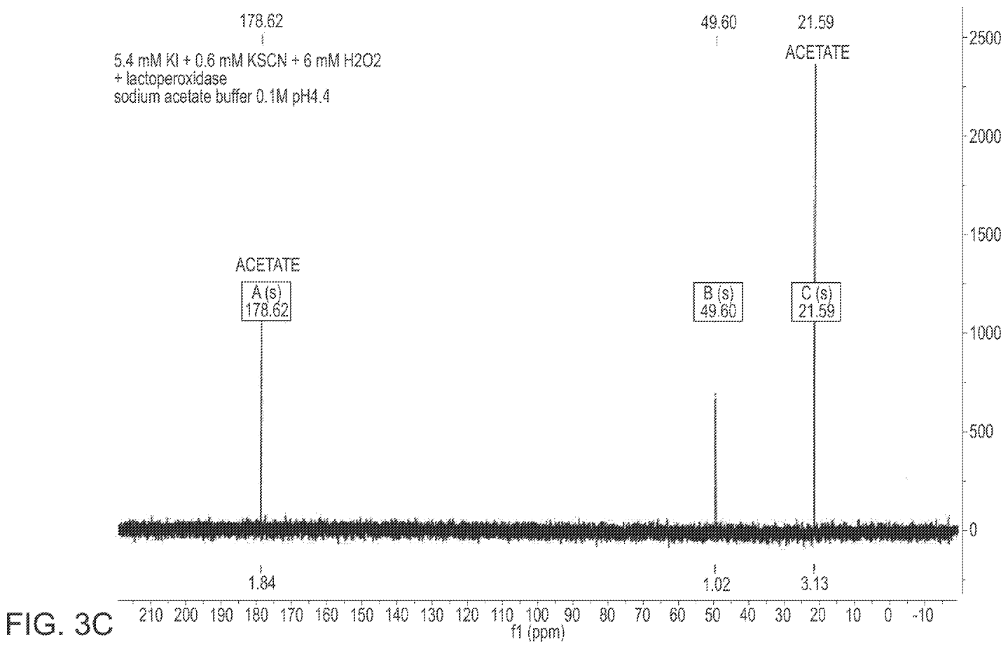

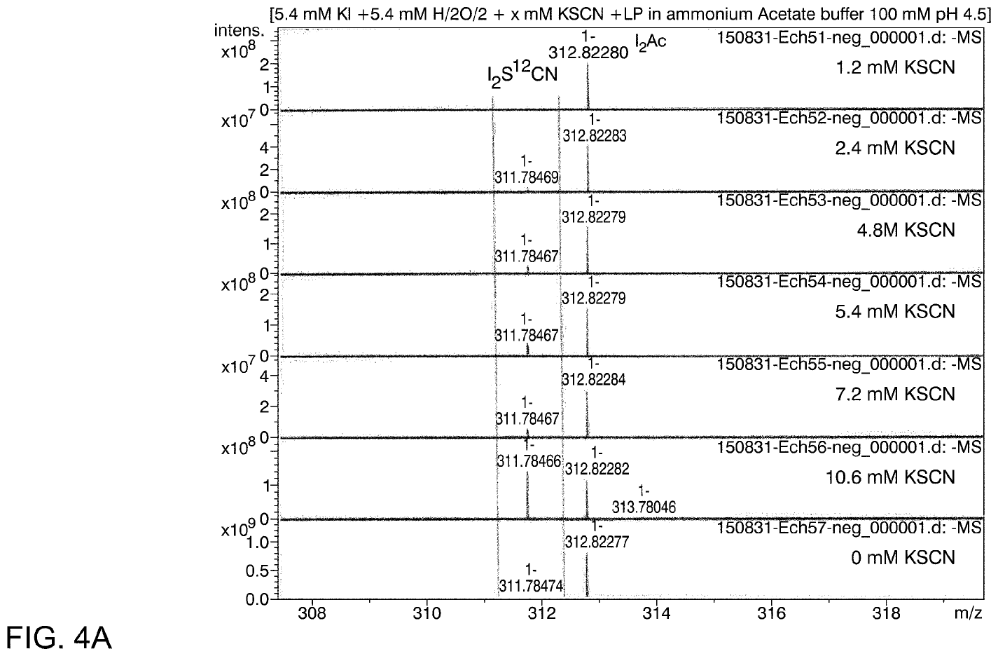

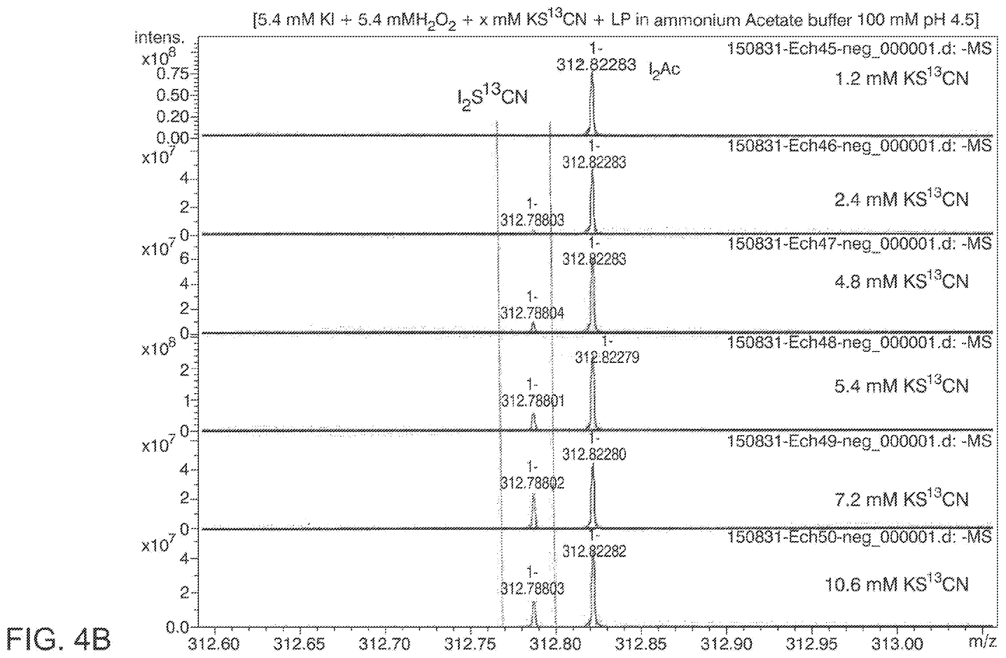

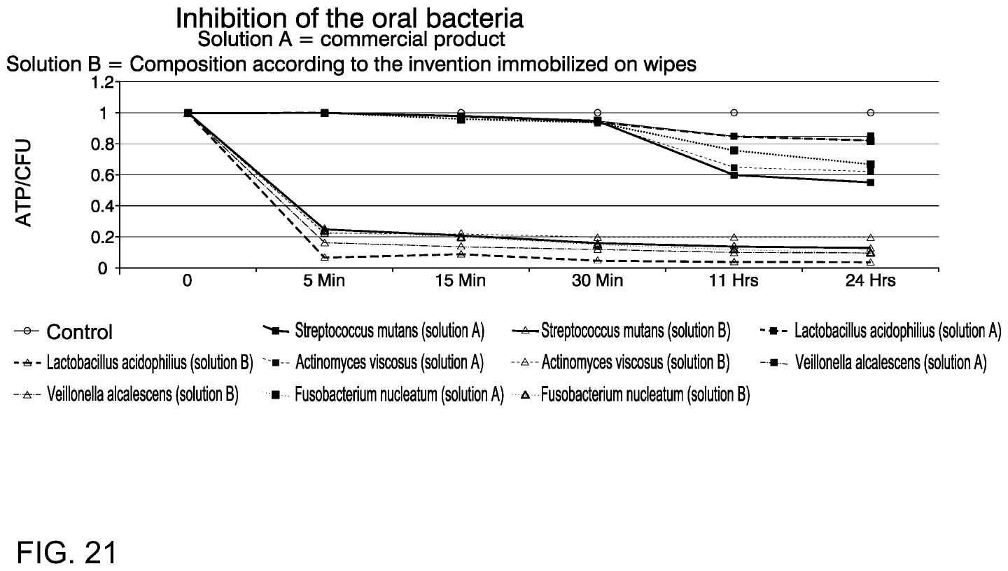

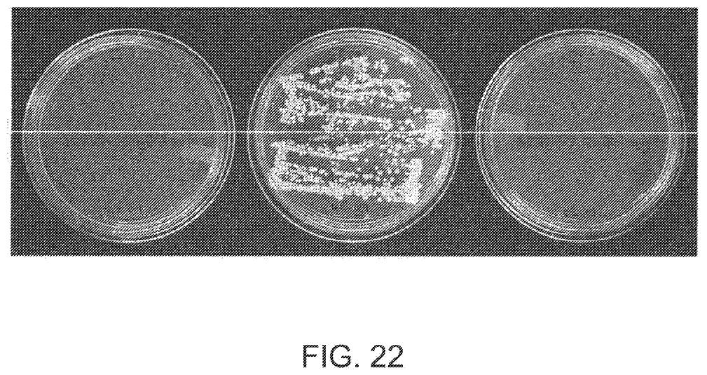

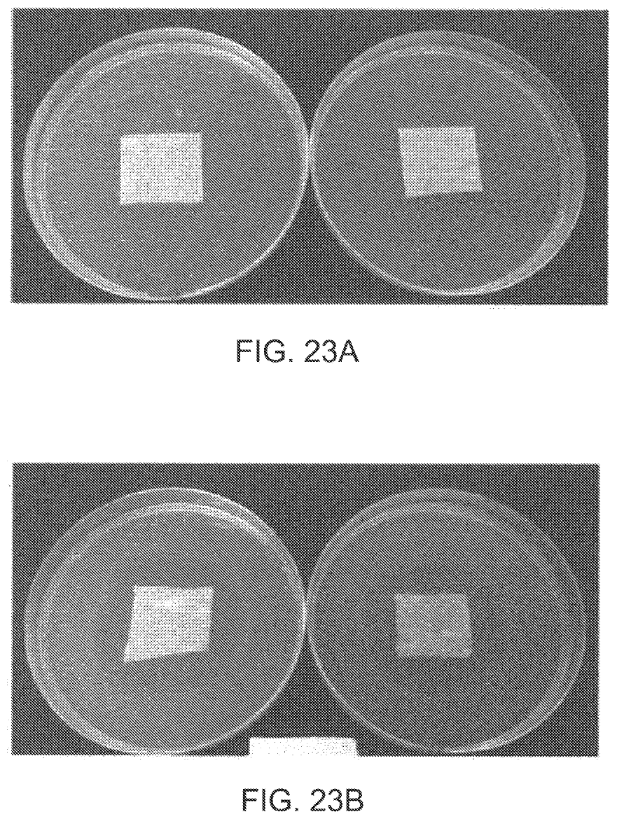

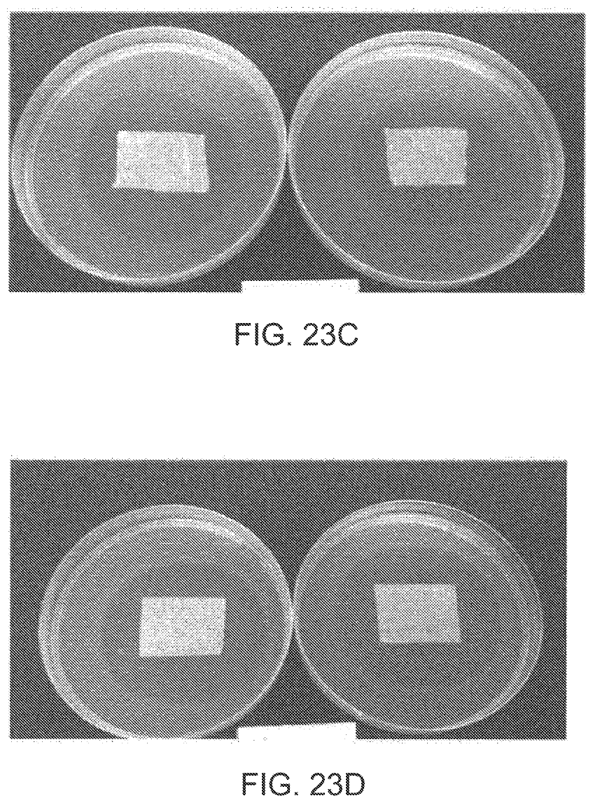

FIG. 21 legend:
Circle-shaped dots: Control
Square-shaped dots: Solution A corresponding to the commercial product (Chlorhexide gluconate 2%)
Triangular-shaped dots: Solution B corresponding to the composition according to the invention, immobilized on wipes

From the curve closest to 0 ATP/CFU at 24 hours of treatment, and going up to the control curve defined by the circles-shaped points, corresponding to 1 ATP/CFU:
Lactobacillus acidophilus in the presence of solution B
Veillonella alcalescens in the presence of solution B
Fusobacterium nucleatum in the presence of solution B
Streptococcus mutans in the presence of solution B
Actinomyces viscosus in the presence of solution B
Streptococcus mutans in the presence of solution A
Actinomyces viscosus in the presence of solution A
Fusobacterium nucleatum in the presence of solution A
Veillonella alcalescens in the presence of solution A
Lactobacillus acidophilus in the presence of solution A

FIG. 22: Action of a solution of I₂SCN⁻ on bioflims (resin). The resin is a material used in the preparation of dental prostheses, obtained by polymerization of organic compounds.

FIG. 22 is a photograph which shows the sterilization of a strip of resin contaminated by Candida albicans ATCC 10231 after immersion in a solution of I₂SCN⁻. The biofilm formed on the resin is totally destroyed after 30 min of contact at ambient temperature. Shown on the left is the control which is a sterile resin strip, in the middle, a strip contaminated by Candida albicans, on the right, a strip contaminated and disinfected with a solution containing 250 uM of I₂SCN⁻ ions

FIGS. 23A-23D: Action of the composition according to the invention immobilized on a fabric.

A composition according to the invention was prepared by bringing together of 5.4 mM of potassium iodide (KI), 1.2 mM of potassium thiocyanate (KSCN), 6.6 mM of hydrogen peroxide (H₂O₂) in the presence of 50 mg/L of lactoperoxidase (LP) (1000 ABTS units per mg) in a sodium citrate buffer 100 mM pH 6.2 (FIG. 23B) or a phosphate buffer 100 mM pH 7.4 (FIG. 23C) or a sodium citrate buffer 100 mM pH 6.2 and lyophilized after preparation and reconstituted in water (FIG. 23D). These compositions were immobilized on a fabric, and the antibacterial activity of these fabrics impregnated with the compositions was tested with respect to E. coli (FIGS. 23B, 23C and 23D).

FIGS. 23A-D illustrate the action which the composition according to the invention has on E. coli (10⁹ CFU/mL). It is apparent that, after 24 h of incubation at 37° C. of 100 μL of E. coli at 10⁹ CFU/mL in a culture medium in a petri dish, the composition immobilized on a fabric prevents the development of the bacterium (apparent halo). It can be seen that the lyophilized composition maintains a bactericidal action equivalent to the other non-lyophilized compositions. The “control” fabric was impregnated with sterile water.

### EXAMPLES

### Example 1: Preparation of Compositions According to the Invention

In general, a certain number of compositions according to the invention were prepared under different conditions:
of buffers/concentrations of buffer/pH;
of pH in the absence of a buffer (in water);
of I/SCN⁻ ratios,
of concentration of peroxidase.

The compositions according to the invention are prepared according to the general protocol described below, said protocol being accessible to the person skilled in the art without further explanation.

A first solution comprising iodide ions (I⁻) at an appropriate molar concentration is prepared. In parallel, a second solution comprising thiocyanate (SCN⁻) ions at an appropriate molar concentration is prepared. In parallel, a third solution of hydrogen peroxide at an appropriate molar concentration (namely the sum of the two preceding molar concentrations) is prepared.

In parallel, a “teabag” comprising lactoperoxidase (LP) is prepared.

The “teabag” is immersed in water or a buffered aqueous solution.

The first two solutions (comprising the iodide ions and the thiocyanates ions, respectively) are added to the water or the aqueous solution comprising the “teabag.”

The third solution (comprising the hydrogen peroxide) is added to the mixture.

After approximately 60 seconds of presence simultaneously of the three solutions (comprising the iodide ions, the thiocyanate ions and the H₂O₂, respectively), and the lactoperoxidase (LP) is removed by means of the teabag.

After removal of the lactoperoxidase (LP), several analyses can be carried out on the products of the oxidation reaction:
¹³C NMR analysis;
Measurement of the oxidizing activity of the —SH groups
Measurement of the oxidizing activity of the —NH₂ groups

In the context of the present application, ¹³C NMR was used for identifying and quantifying the ions. In addition, it was used to confirm that no hypothiocyanite ion (OSCN⁻) was detectable in the compositions according to the invention.

The presence of the I₂SCN⁻ and I(SCN)₂⁻ Ion mixture is demonstrated by the presence of a characteristic peak at approximately 49 to 50.5 ppm. The absence of the hypothiocyanite ions is demonstrated by the absence of peaks at approximately 127 to 128 ppm

This absence of hypothiocyanite ions was also revealed by ionic chromatography.

Analysis of the oxidation of the SH et NH₂ functions; The analysis of the oxidation of the NH₂ functions is carried out by oxidation of TMB (tetramethylbenzidine).
The analysis of the oxidation of the SH function is carried out by oxidation of TNB (5-thio-2-nitrobenzoic acid) Into DTNB (5,5′-dithiobis-(2-nitrobenzoic acid)).
Testing of the compositions on microorganisms

### Example 1.1: Influence of the Buffers/pH/Concentrations of Buffer

The recapitulative table of the tests is presented below:

TABLE 1

| | | NMR | | | | | |
|---|---|---|---|---|---|---|---|
| Oxidation | Oxidation | (S13CN) | Position | New | NMR (relative intensity) | | |
| SH - μM/L | NH2 - μM/L | SCN- | OSCN- | Peak | SCN- | OSCN- | New peak |

2.4 mM KSCN + 2.4 mM H₂O₂ + LP

| | | | | | | | | | |
|---|---|---|---|---|---|---|---|---|---|
| 1 | Tap water | 374 | 0 | 133.48 | 127.71 | no | 320 | 320 | NA |
| 2 | Phosphate buffer - 100 mM - pH 7.4 | — | — | 133.48 | 127.71 | no | — | — | NA |

5.4 mM KI + 1.2 mM KSCN + 6.6 mM H₂O₂ + LP

| | | | | | | | | | |
|---|---|---|---|---|---|---|---|---|---|
| 3 | Tap water | 442 | 504 | 133.48 | no | no | 160 | NA | NA |
| 4 | Na acetate buffer - 500 mM - pH 4.4 | 864 | 1094 | no | no | 49.8 | NA | NA | 1425 |
| 5 | Na acetate buffer - 100 mM - pH 4.4 | 829 | 1080 | no | no | 49.63 | NA | NA | 1650 |
| 6 | Na acetate buffer - 10 mM - pH 4.4 | 827 | 1000 | no | no | 49.58 | NA | NA | 1475 |
| 7 | Na acetate buffer - 1 mM - pH 4.4 | 365 | 164 | 133.48 | no | 49.62 | 110 | NA | 250 |
| 8 | NH₄ acetate buffer - 500 mM - pH 4.5 | 1191 | 1303 | no | no | 50.05 | NA | NA | 1800 |
| 9 | NH₄ acetate buffer - 100 mM - pH 4.5 | 964 | 1150 | no | no | 49.69 | NA | NA | 1700 |
| 10 | NH₄ acetate buffer - 10 mM - pH 4.5 | 962 | 1187 | no | no | 49.59 | NA | NA | 1550 |
| 11 | NH₄ acetate buffer - 1 mM - pH 4.5 | 576 | 539 | 133.48 | no | 49.6 | 150 | NA | 660 |
| 12 | Na acetate buffer - 500 mM - pH 5.6 | 1172 | 1074 | no | no | 50.51 | NA | NA | 1725 |
| 13 | Na acetate buffer - 100 mM - pH 5.6 | 983 | 1056 | no | no | 49.71 | NA | NA | 1800 |
| 14 | Na acetate buffer -10 mM - pH 5.6 | 964 | 666 | 133.48 | no | 49.61 | 170 | NA | 200 |
| 15 | Na acetate buffer - 1 mM - pH 5.6 | 659 | 684 | 133.48 | no | 49.61 | 160 | NA | 290 |
| 16 | Citrate buffer - 500 mM - pH 6.2 | 1260 | 1375 | no | no | 51.11 | NA | NA | 1175 |
| 17 | Citrate buffer - 100 mM - pH 6.2 | 948 | 1000 | no | no | 49.97 | NA | NA | 1125 |
| 18 | Citrate buffer - 10 mM - pH 6.2 | 781 | 828 | no | no | 49.64 | NA | NA | 852 |
| 19 | Phosphate buffer - 500 mM - pH 7.4 | 1035 | 1552 | no | no | 51.16 | NA | NA | 1035 |
| 20 | Phosphate buffer -100 mM - pH 7.4 | 767 | 900 | no | no | 50.36 | NA | NA | 1650 |
| 21 | Phosphate buffer - 10 mM - pH 7.4 | 475 | 502 | 133.48 | no | no | 94 | NA | NA |
| 22 | Phosphate buffer - 1 mM - pH 7.4 | 271 | 284 | 133.48 | no | no | 205 | NA | NA |
| 23 | Carbonate buffer - 500 mM - pH 9.2 | 328 | 87 | 133.48 | no | no | 185 | NA | NA |
| 24 | Carbonate buffer - 100 mM - pH 9.2 | 248 | 106 | 133.48 | no | no | 225 | NA | NA |
| 25 | Carbonate buffer - 10 mM - pH 9.2 | 214 | 212 | 133.48 | no | no | 230 | NA | NA |

First, it is specified that no hypothiocyanite ion (OSCN⁻) is detected for all of the compositions in which the two ions, I⁻ and SCN⁻, were introduced (tests 3-25), while such ions form in the absence of I⁻ (tests 1-2).

As explained in the introduction of Example 1, the peak in the vicinity of 50 ppm in NMR is characteristic of the new species identified, namely I₂SCN⁻ and I(SCN)₂⁻. Moreover, its intensity reveals the quantity of new species formed.

This peak is observed when the pH of the solution is from 4.4 to 7.4. If the pH is less than 4.4, hydrolysis of the thiocyanate occurs.

When the assayed quantity of oxidizing molecules is high and the peak at 49-50 ppm can be seen to appear, a peak associated with the thiocyanate is no longer observed, which indeed indicates its participation in the reaction.

### Example 1.2: Influence of the pH on the Water Base (with LP)

The recapitulative table of the tests is presented below:

TABLE 2

| | | NMR | | | | | |
|---|---|---|---|---|---|---|---|
| Oxidation | Oxidation | (S13CN) | Position | New | NMR (relative intensity) | | |
| SH - μM/L | NH₂ - μM/L | SCN- | OSCN- | Peak | SCN- | OSCN- | New peak |

5.4 mM KI + 1.2 mM KSCN + 6.6 mM H₂O₂ + LP

| | | | | | | | | | |
|---|---|---|---|---|---|---|---|---|---|
| 26 | Water - pH 4.4 | 1130 | 1622 | no | no | 49.61 | NA | NA | 1300 |
| 27 | Water - pH 5.5 | 1010 | 821 | no | no | 49.62 | NA | NA | 740 |
| 28 | Water - pH 6.5 | 644 | 1098 | 133.48 | no | no | 120 | NA | NA |
| 29 | Water - pH 7.5 | 505 | 567 | 133.48 | no | no | 210 | NA | NA |

Here again, in the presence of iodide ion, no hypothiocyanate ion (OSCN⁻) is detected, irrespective of the compound.

When the unbuffered aqueous solution is at a pH of less than 6.5, it is observed that the new peak is detected, which is characterized by a shift in NMR (49.6 ppm), which is correlated with an increased capacity to oxidize the SH and NH₂ groups.

The new peak is observed at acidic pH values: 5.5 and 4.4, with a nearly doubled intensity at pH 4.4.

In contrast, at higher pH values, the new peak is not observed.

### Example 1.3: Influence of the I⁻/SCN⁻ Ratio

The recapitulative table of the tests is presented below:

TABLE 3

| | | NMR | | | | | | | | |
|---|---|---|---|---|---|---|---|---|---|---|
| | | (S13CN) | Position | New | Other | NMR (relative intensity) | | | | |
| | | SCN⁻ | OSCN⁻ | Peak | Peak | SCN⁻ | OSCN- | New peak | Other peaks | |
| 30 | 9:1 5.4 mM KI + 0.6 mM KSCN + | no | no | 49.6 | no | NA | NA | 740 | NA | NA |
| | 6 mM H₂O₂ Na acetate | | | | | | | | | |
| | buffer pH 4.4 0.1M | | | | | | | | | |
| 31 | 1:1 5.4 mM KI + 5.4 mM KSCN + | 133.47 | no | 49.65 | 124.63/111.94 | 560 | NA | 190 | 800 | 130 |
| | 10.8 mM H₂O₂ Na acetate | | | | | | | | | |
| | buffer pH 4.4 0.1M | | | | | | | | | |
| 32 | 0.54:1 5.4 mM KI + 10 mM KSCN + | 133.48 | no | no | 124.63/111.94 | 1400 | NA | NA | 1750 | 150 |
| | 15.4 mM H₂O₂ Na acetate | | | | | | | | | |
| | buffer pH 4.4 0.1M | | | | | | | | | |
| 33 | 1:1 1.2 mM KI + 1.2 mM KSCN + | 133.48 | no | no | 124.63/111.93 | 75 | NA | NA | 125 | 105 |
| | 2.4 mM H₂O₂ Na acetate | | | | | | | | | |
| | buffer pH 4.4 0.1M | | | | | | | | | |
| 34 | 0.27:1 1.2 mM KI + 5.4 mM KSCN + | 133.48 | no | no | 124.63 | 825 | NA | NA | 750 | NA |
| | 6.6 mM H₂O₂ Na acetate | | | | | | | | | |
| | buffer pH 4.4 0.1M | | | | | | | | | |
| 35 | 1:2 5.4 mM KI + 2.7 mM KSCN + | 133.48 | no | 49.62 | no | 100 | NA | 950 | NA | NA |
| | 8.1 mM H₂O₂ Na acetate | | | | | | | | | |
| | buffer pH 4.4 0.1M | | | | | | | | | |
| 36 | 4.5 5.4 mM KI + 1.2 mM KSCN + | no | no | 49.69 | no | NA | NA | 1700 | NA | NA |
| | 6.6 mM H₂O₂ + ammonium acetate | | | | | | | | | |
| | buffer 100 mM pH 4.5 | | | | | | | | | |

Here again, in the presence of iodide ion, no hypothiocyanate ion (OSCN⁻) is detected, irrespective of the compound.

The new peak is observed at I⁻/SCN⁻ ratios from 9/1 to strictly greater than 1/1.

In contrast, no peak is observed at I⁻/SCN⁻ ratios from 0.27/1 to 1/1.

### Example 2: Efficacy of a Composition According to the Invention

A composition according to the invention was prepared by bringing together of potassium iodide (KI) 5.4 mM, 1.2 mM of potassium thiocyanate (KSCN), 6.6 mM of hydrogen peroxide (H₂O₂) in an ammonium acetate buffer 100 Mm, pH 4.5 in the presence of 50 mg/L of lactoperoxidase (LP) (1000 ABTS units per mg) according to the protocol described in Example 1.

This compound was compared with the compounds of the prior art manufactured by bringing together of:
either potassium iodide (KI), lactoperoxidase (LP) and hydrogen peroxide (H₂O₂);
or potassium thiocyanate (KSCN), lactoperoxidase (LP) and hydrogen peroxide (H₂O₂).

The results are given in the table below:

TABLE 4

| | KSCN + KI | KSCN | KI |
|---|---|---|---|
| Colletotrichum lindemuthanium | ++++ | − | − |
| Fusarium avenaceum | +++ | − | − |
| Septoria tritici | + | − | − |
| Verticillium dahliae | ++++ | − | − |
| Phytophthom infestans | +++ | − | − |
| Pythium ultimum | ++++ | − | − |
| Colletotrichum musae | ++++ | 0 | 0 |
| Penicillium italicum | ++++ | 0 | ++++ |
| Penicillium digitatum | ++++ | + | ++++ |
| Botrytis cinerea | ++++ | − | − |
| Penicillium expansum | ++++ | − | − |
| Pectobacterium atrosepticum | ++++ | + | + |
| Pseudomonas syringae pv syringae | ++++ | + | 0 |
| Pectobacterium atrosepticum | ++++ | + | 0 |
| Erwynia amylovora | ++++ | + | 0 |
| Pseudomonas syringae pv. tomato | ++++ | +++ | + |
| Clavibacter michiganensis | ++++ | ++++ | +++ |
| subsp. michiganensis | | | |
| Kocuria rhizolia | ++++ | + | + |
| Staphylococcus aureus | ++++ | ++ | ++ |
| Enterobacter gergoviae | ++++ | + | + |
| Escherichia coli | ++++ | + | + |
| Klebsiella pneumoniae | ++++ | + | + |
| Pseudomonas aeruginosa | ++++ | + | + |
| Pseudomonas fluorescent | ++++ | + | + |
| Pseudomonas putita | ++++ | + | + |
| Aspergillus niger | ++++ | − | + |
| Penicillium pinophilum | ++++ | + | ++++ |
| Candida albicans | ++++ | + | ++ |
| Xylella fastidiosa subsp. fastidiosa | ++++ | − | − |
| Xylella fastidiosa subsp. multiplex | ++++ | − | − |
| Xylella fastidiosa subsp. pauca | ++++ | − | − |

% inhibition:
++++: 79-100%
+++: 60-78%
++: 41-59%
+: 0-40%
0: no inhibition
−: not tested

One observes that a composition according to the invention has an often much greater activity on all the microorganisms tested.

### Example 3: Oxidizing Power of a Composition According to the Invention

A composition according to the invention was prepared by bringing together of potassium iodide (KI) 5.4 mM, 1.2 mM of potassium thiocyanate (KSCN), 6.6 mM of hydrogen peroxide (H₂O₂) in an ammonium acetate buffer 100 mM, pH 4.5 in the presence of 50 mg/L of lactoperoxidase (LP) (1000 ABTS units per mg) according to the protocol described in Example 1.

The time of contact of the different solutions was set at 1 minute.

The oxidizing power was then measured on the SH functions by the method of oxidation of TNB (5-thio-2-nitrobenzoic acid) into DTNB (5,5′-dithiobis-(2-nitrobenzoic acid)), after the following storage times: 1, 3, 5, 10, 15, 20, 30, 60 and 120 minutes.

The time T=0 corresponds to the time when the lactoperoxidase (LP) is removed.

The results are given in the table below:

TABLE 5

| Time | |
|---|---|
| (minutes) | Oxidation -SH |
| 0 | 516.54 |
| 3 | 567.64 |
| 5 | 586.4 |
| 10 | 586.7 |
| 15 | 587.02 |
| 20 | 584.68 |
| 30 | 581.78 |
| 60 | 701.58 |
| 120 | 684.7 |

It is noted that the oxidizing activity increases with the storage time.

In addition, after 60 minutes, the oxidizing power is stable.

### Example 4: Stability of a Composition According to the Invention

A composition according to the invention was prepared by bringing together of potassium iodide (KI) 5.4 mM, 1.2 mM of potassium thiocyanate (KSCN), 6.6 mM of hydrogen peroxide (H₂O₂) in an ammonium acetate buffer 100 mM, pH 4.5 in the presence of 50 mg/L of lactoperoxidase (LP) (1000 ABTS units per mg) according to the protocol described in Example 1.

This composition was distributed in 6 flasks. The flasks are then opened (1 per month), and the bactericidal activity on Candida albicans is tested.

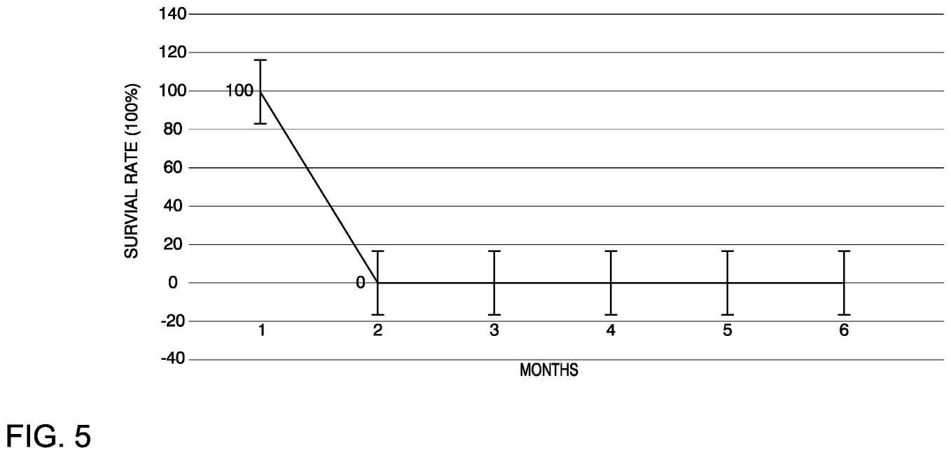

The graph illustrating the variation in the survival rate of the Candida albicans as a function of time, evaluated over a period of 6 months, is presented in FIG. 5

It is observed that no measurable decrease in the activity is detected during the period of 6 months.

### Example 5: Test of a Short Time of Contact with Candida albicans (5 Minutes)

A composition according to the invention was prepared by bringing together of potassium iodide (KI) 5.4 mM, 1.2 mM potassium thiocyanate (KSCN), 6.6 mM hydrogen peroxide (H₂O₂) in an ammonium acetate buffer 100 Mm, pH 4.5 in the presence of 50 mg/L of lactoperoxidase (LP) (1000 ABTS units per mg) according to the protocol described in Example 1.

The bactericidal activity on Candida albicans was tested, for the following times of contact with Candida albicans: 5 minutes or 30 minutes.

After a contact time of 5 minutes or 30 minutes with the composition (or the control), an inoculation of a culture medium takes place.

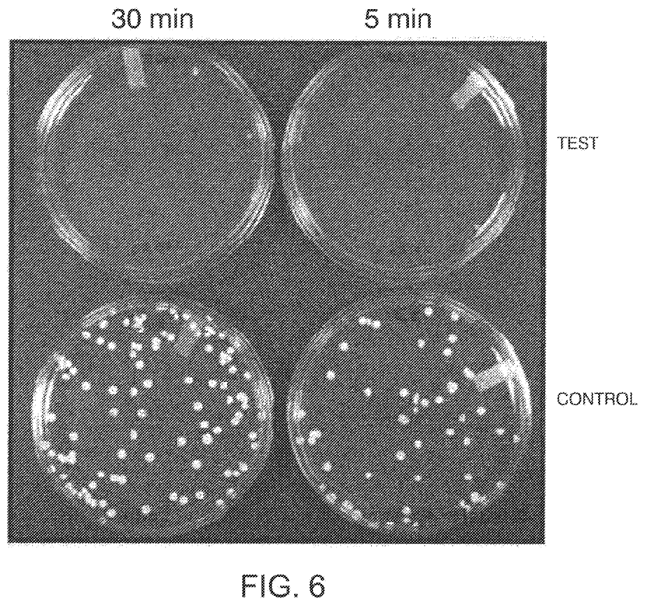

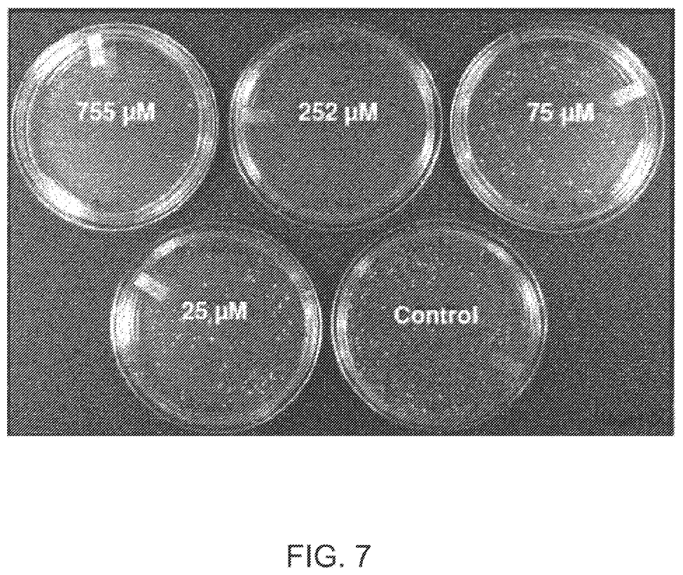

The results of these tests are illustrated in FIG. 6 (photographs of the culture media after contact versus control): whether the contact time is 30 minutes (left portion of the figure) or 5 minutes (right portion of the figure), the visual result is the same: no colony of Candida albicans resists the composition according to the invention.

### Example 6: Efficacy with a Decreased Concentration of Oxidizing Agents

A composition according to the invention was prepared by bringing together of potassium iodide (KI) 5.4 mM, 1.2 mM of potassium thiocyanate (KSCN), 6.6 mM of hydrogen peroxide (H₂O₂) in an ammonium acetate buffer 100 mM, pH 4.5 in the presence of 50 mg/L of lactoperoxidase (LP) (1000 ABTS units per mg) according to the protocol described in Example 1.

This composition was distributed in 5 flasks according to different dilutions in decreasing order: 755 μm, 252 μm, 75 μm, 25 μm. A control solution was also prepared.

After a contact time of 5 minutes with the 6 different compositions (5 dilutions and the control), an inoculation of a culture medium takes place.

The results are presented in FIG. 7: even at a concentration of 25 μm, it is observed that the number of colonies formed is greatly reduced in comparison to the control. In addition, up to 252 μm, the activity is not reduced.

### Example 7: Selectivity of a Composition According to the Invention Streptococcus mutans/Streptococcus salivarius

A composition according to the invention was prepared by bringing together of potassium iodide (KI) 5.4 mM, 1.2 mM of potassium thiocyanate (KSCN), 6.6 mM of hydrogen peroxide (H₂O₂) in an ammonium acetate buffer 100 mM, pH 4.5 in the presence of 50 mg/L of lactoperoxidase (LP) (1000 ABTS units per mg) according to the protocol described in Example 1.

The composition is tested with regard to its bactericidal effect on Streptococcus mutans (cariogenic bacterium) and on Streptococcus salivarius (commensal bacterium).

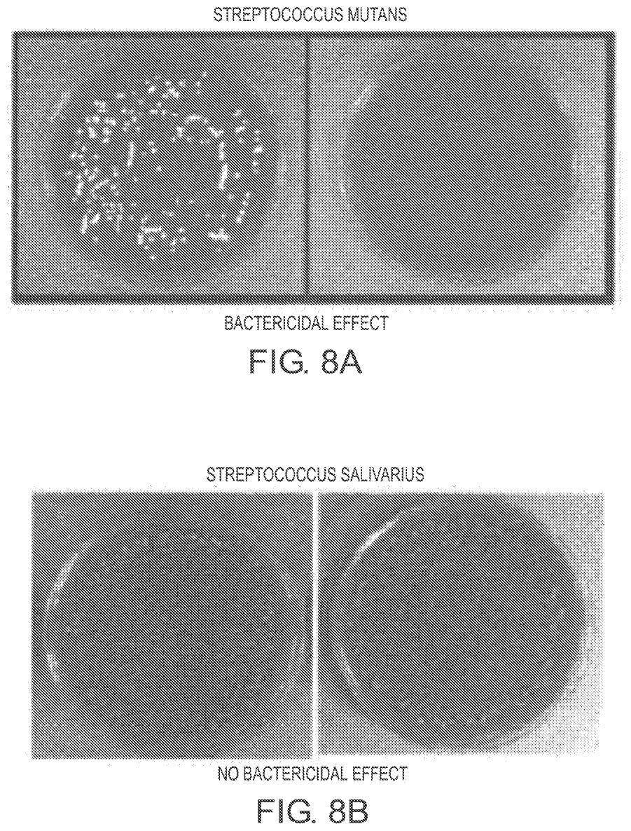

The results are presented in FIGS. 8A and 8B: the composition according to the invention has a bactericidal effect on Streptococcus mutans (FIG. 8A) and no effect on Streptococcus salivarius (FIG. 8B).

### Example 8: Activity with Respect to the Biofilms

A composition according to the invention according to the protocol described in Example 1.

This composition was diluted 3-fold and has a concentration of I₂SCN⁻ ions of 250 μM. This composition is called “solution B.”

This composition was put in contact with different biofilms organized bacteria versus control and versus another other commercial composition which is Chlorexhidine gluconate 2.0% (“solution A”).

The compositions were put in contact with the biofilm of the different bacteria for variable durations: 5 minutes, 15 minutes, 30 minutes, 11 hours and 24 hours. This contact occurs by immersion of the strips containing the bacterial biofilm in a solution of the composition or in a solution of chlorhexidine gluconate 2% or as control in an aqueous solution (see hereafter the details of the technique applied for Candida albicans)

The activity with respect to the following bacteria was tested: Lactobacillus acidophilus, Veillonella alcalescens, Streptococcus mutans, Actinomyces viscosus, and Fusobacterium nucleatum.

The results are presented in FIG. 21: the composition according to the invention is active against all the bacteria tested, starting at 5 minutes of contact, in contrast to the solution of Chlorhexidine gluconate 2% which has an action which is less effective and starts only after 11 hours of contact. The control obviously has no action.

Candida albicans biofilms

Protocol of Immobilization of Biofilms of Candida albicans on Resin and Titanium.

A) Resin: Material Used in the Fabrication of Dental Prostheses, Obtained by Polymerization of Organic Compounds

The pieces of resin are stored in sodium azide (0.5 g/500 mL) in order to be disinfected. This operation occurs under sterile conditions.

Transfer 3 pieces of resin into a pot.

Wash 3× in 60 mL of H₂O for 5 min with stirring.

Rinse a last time with 60 mL of Sabouraud liquid for 5 min with stirring.

Transfer each piece of resin into a 10 mL round-bottom tube.

Prepare a suspension of Candida albicans at 10⁶ bi/mL in 10 mL of Sabouraud medium.

Prepare the 3 reaction tubes as indicated in the table below.

| Preparation of the biofilms on resin | | |
|---|---|---|
| (composition of the tubes on D₁). | | Suspension |
| Sabouraud liquid | | of C.a. |
| (mL) | | (mL) |
| Control− | 4 | — |
| Control+ | 3.6 | 0.4 |
| Test | 3.6 | 0.4 |

Incubate for 24 to 48 h in a Rotator™ (3 rotations per minute).
After incubation, from each of the 3 tubes, transfer 1 mL of supernatant into a cuvette. Measure the absorbance at 600 nm.
Suction the culture media in the 3 tubes.
Wash 3× in phosphate buffer.
Prepare a phosphate buffer solution containing glucose (2 g/100 mL).
Using this buffer, prepare a solution of I₂SCN⁻.
Prepare the 3 reaction tubes as indicated in the table below.

| Preparation of the biofilms on resin | | |
|---|---|---|
| (composition of the tubes on D₂). | | |
| Phosphate buffer + glucose | | I₂SCN⁻ |
| (mL) | | (mL) |
| Control− | 4 | — |
| Control+ | 4 | — |
| Test | — | 4 |

Incubate for 30 min.
Inoculate each face of the resin strips successively in 4 Petri dishes.
Incubate for 24 to 48 h at 37° C.

B) Titanium

This operation occurs under sterile conditions.

Weigh 500 mg of titanium in 3 different tubes.

Prepare a suspension of Candida albicans at 10⁶ bl/mL in 10 mL of Sabouraud medium.

Prepare the 3 reaction tubes as indicated in the table below.

| Preparation of the biofilms on titanium | | |
|---|---|---|
| (composition of the tubes on D₁). | | Suspension |
| Liquid Sabouraud | | of C.a. |
| (mL) | | (mL) |
| Control− | 4 | — |
| Control+ | 3.6 | 0.4 |
| Test | 3.6 | 0.4 |

Incubate for 24 h to 48 h in a Rotator™ (3 rotations per minute).
After incubation, allow settling for exactly 10 min.
From each of the 3 tubes, transfer 1 mL of supernatant into a cuvette. Measure the absorbance at 600 nm.
Suction the culture media in the 3 tubes.
Wash 3 times in phosphate buffer.
Prepare a solution of I₂SCN⁻.
Prepare a phosphate buffer solution containing glucose (2 g/100 mL).
Prepare the 3 reaction tubes as indicated in the table below.

| Preparation of the biofilms on titanium | | |
|---|---|---|
| (composition of the tubes on D₂). | | |
| Phosphate buffer + glucose | | I₂SCN⁻ |
| (mL) | | (mL) |
| Control− | 4 | — |
| Control+ | 4 | — |
| Test | — | 4 |

Suction the supernatant
   Assay with MTT (*)

The living blastoconidia transform the MTT-tetrazolium into MTT-formazan which absorbs at 570 nm (Levitz & Diamond, 1985).

The different steps of the procedure are detailed below:
Prepare a suspension of Candida albicans of 10⁶ bl/mL in 10 mL of phosphate buffer containing glucose (2 g/100 mL)
(*) MTT: 3-(4,5-dimethylthiazol-2-yl)-2,5-diphenyltetrazolium bromide
Prepare the 4 reaction tubes as indicated in the table below.

| Preparation of the tubes for | | | | |
|---|---|---|---|---|
| the assay with MTT. Blank | | Control− | Control+ | Test |
| Suspension of C.a. (mL) | — | — | — | — |
| MTT (mL) | 1.5 | 1.5 | 1.5 | 1.5 |
| Phosphate buffer + glucose (mL) | 3 | 3 | 3 | 3 |

Shown in FIG. 22 are:

Control − (on the left): sterile resin strip.

Control + (in the middle): contaminated strip.

Test (on the right): strip contaminated and disinfected with the solution A

One observes the sterilization of a resin strip contaminated by Candida albicans ATCC 10231 after immersion in a solution containing 250 μM of I₂SCN⁻ ions. The biofilm formed on the resin was totally destroyed after 30 min of contact at ambient temperature.

The action of a solution of I₂SCN⁻ on biofilms on (titanium) is measured by assay of the Candida biofilms with MTT.

The solution containing 250 μM of ions I₂SCN⁻ makes it possible to destroy ˜70% of the biofilms formed for 24 to 48 h.

The results obtained are collected below:

| Control− | Control+ | Test |
|---|---|---|
| 0.003 | 0.949 | 0.319 |

### Example 9: Stability and Activity of a Composition According to the Invention for the Application to the Problems of Contamination of Paints and Resins

A composition according to the invention was prepared by bringing together of potassium iodide (KI) 5.4 mM, 1.2 mM of potassium thiocyanate (KSCN), 6.6 mM of hydrogen peroxide (H₂O₂) in an ammonium acetate buffer 100 mM, pH 4.5 in the presence of 50 mg/L of lactoperoxidase (LP) (1000 ABTS units per mg) according to the protocol described in Example 1.

The samples of paints and resins were contaminated by several series of microorganisms such as bacteria, yeasts and molds and mixtures thereof.

The tests were carried out by adding the composition according to the invention from the start. After a waiting time of 24 hours, the resin and paint samples were inoculated with a suspension of a mixture of microorganisms so as to reach a contamination level of 1,000,000 CFU/mL. The mixtures of the microorganisms consisted of bacteria, yeasts and molds such as:
bacteria: Pseudomonas fluorescent ATCC 9721, Pseudomonas aeruginosa ATCC 10145, Bacillus subtilis ATCC 6984, Proteus vulgaris ATCC 9920;
yeasts: Candida tropicalis ATCC 750, Kluyveromyces fragilis ATCC 8554, Candida pseudotropicalis ATCC 4135;
molds: Aspergillus niger ATCC 9642, Aspergillus flavus ATCC 9643, Penicillium pinophilum ATCC 9644.

After contact times of 1, 2 and 7 days, the microbiological analyses were carried out. They are summarized in table 6 below:

TABLE 6

| | 1 day | 2 days | 7 days |
|---|---|---|---|
| Mixture A: 1,000,000 CFU/mL | | | |
| Pseudomonas fluorescent | ++++ | ++++ | ++++ |
| Pseudomonas aeruginosa | ++++ | ++++ | ++++ |
| Candida tropicalis | ++++ | ++++ | ++++ |
| Aspergillus niger | ++++ | ++++ | ++++ |
| Mixture B: 1,000,000 CFU/mL | | | |
| Pseudomonas aeruginasa | ++++ | ++++ | ++++ |
| Kluyveromyces fragilis | ++++ | ++++ | ++++ |
| Penicillium pinophilum | ++++ | ++++ | ++++ |

% inhibition
++++ 79-100%
+++ 60-78%
++ 41-59%
+ 0-40%
0 No inhibition

One notes that there is no contamination, irrespective of the sample. This confirms that the composition according to the invention has an activity that stops the growth of the microorganisms in paints and resins.

### Example 10: Activity of a Composition According to the Invention for the Problems of Contamination of Soils, Production Materials and Dental, Surgical Instruments, Etc.

In industrial processes, a cleaning referred to as CIP (Cleaning In Place) is applied, which consists in applying disinfectants at high temperature after use of the equipment.

The same applies to the instruments used in dentistry practices, in hospitals, instruments which are cleaned after their use by sterilization in an oven at high temperature.

A composition according to the invention was prepared by bringing together of potassium iodide (KI) 5.4 mM, 1.2 mM of potassium thiocyanate (KSCN), 6.6 mM of hydrogen peroxide (H₂O₂) in an ammonium acetate buffer 100 mM, pH 4.5 in the presence of 50 mg/L of lactoperoxidase (LP) (1000 ABTS units per mg) according to the protocol described in Example 1.

It was demonstrated that this composition was capable of eliminating the microorganisms responsible for contaminations of this industrial equipment and other equipment and had the advantage that it could be used at ambient temperature.

### Example 11: Activity of a Composition According to the Invention for the Problems of Contamination During the Cicatrization of Injuries in Humans and Animals

The resistance of microorganisms to the antibiotics is an increasing problem and seriously complicates the cicatrization of wounds caused by an injury or a burn of the skin. These microorganisms are capable of increasing the inflammatory process.

A composition according to the invention was prepared according to the protocol described in Example 1 and was diluted until a composition comprising 250 μM of I₂SCN⁻ was obtained.

This composition was tested on microorganisms which are resistant to the current antibiotics.

More precisely, the use of impregnated fabrics showed the potential, when said fabrics were applied to the wound, of having an antibacterial activity against bacterial species that are resistant to different antibiotics.

The results described in the table below show that the composition according to the invention which was prepared according to the protocol described in Example 1 is very effective against these microorganisms even if its concentration of I₂SCN⁻ ions is 250 μM.

TABLE 7

| Bacteria | | 1 day | 2 days | 7 days |
|---|---|---|---|---|
| Burkholderia multivorans | multiresistant | ++++ | ++++ | ++++ |
| Pseudomonas aeruginosa | multiresistant | ++++ | ++++ | ++++ |
| Stenotrophomonas maltophilia | multiresistant | ++++ | ++++ | ++++ |
| Pandoraea apista | multiresistant | ++++ | ++++ | ++++ |
| Achromobacter denitrificans | multiresistant | ++++ | ++++ | ++++ |
| Staphylococcus aureus | multiresistant | ++++ | ++++ | ++++ |
| Enterococcus faecium | multiresistant | ++++ | ++++ | ++++ |
| Enterococcus faecalis | multiresistant | ++++ | ++++ | ++++ |
| Yeasts | | | | |
| Malassezia pachydermatis | multiresistant | ++++ | ++++ | ++++ |

### Example 12: Activity of a Composition According to the Invention Against the Microorganisms Responsible for Respiratory Diseases in Horses and in Humans

A composition according to the invention was prepared according to the protocol described in Example 1 and was tested on microorganisms responsible for respiratory diseases in horses and in humans.

The growth curves between the exponential phase and the stationary growth phase allowed us to select the ideal conditions which corresponded to a concentration of 1,000,000 spores/mL. A composition according to the invention was prepared according to the protocol described in example 1 and showed an efficacy against the microorganisms responsible for respiratory diseases in horses.

The microorganisms were the following: Rhodococcus equi ATCC 25729—Streptococcus equi subsp equi ATCC 53185 and Streptococcus equi subsp zooepidemicus ATCC 43079. They are responsible for respiratory diseases in horses.

Taking into account the growth time of these microorganisms, the tests were carried out after 48 hours and 120 hours of growth of the microorganism.

In all the mixtures, all the hydrogen peroxide is consumed. The thiocyanate and the iodine are consumed in identical proportions.

The composition prepared according to the protocol described in Example 1 was shown to be effective in the inhibition of the microorganisms after a contact time of 5 minutes. 4 concentrations of I₂SCN⁻ in the ion composition were used at different dilutions. Each number represents the results of 3 independently performed experiments.

The percentages of in vitro inhibition of the microorganisms were measured after a contact time of 5 minutes.

Controls were run with solutions without enzyme with only the substrates 5.4 mM KI+2.2 mM KSCN, on the one hand, and with 6.6 mM of H₂O₂. These solutions showed an absence of efficacy on the microorganisms (see table 8 below).

TABLE 8

| | Before | | | | | | Streptococcus | Streptococcus |
|---|---|---|---|---|---|---|---|---|
| | enzymatic | | Rhodococcus | Rhodococcus | Streptococcus | Streptococcus | equi | equi |
| | reaction | | equi | equi | equi | equi | zooepidemicus | zooepidemicus |
| LPO | KI/KSCN/ | | % inhibition | % inhibition | % inhibition | % inhibition | % inhibition | % inhibition |
| U/mL | H202 mM/L | Dilution | at 48 h | at 120 h | at 48 h | at 120 h | at 48 h | at 120 h |
| 50 | 5.4/1.2/6.6 | ⅓− | 86 | 88 | 82 | 87 | 92 | 97 |
| | | ⅕− | 82 | 84 | 80 | 86 | 92 | 94 |
| | | 1/10− | 81 | 83 | 60 | 62 | 90 | 94 |
| | 3.6/0.8/4.4 | ⅓− | 86 | 88 | 65 | 69 | 95 | 98 |
| | | ⅕− | 86 | 88 | 60 | 61 | 74 | 77 |
| | | 1/10− | 63 | 70 | 65 | 69 | 44 | 48 |
| | 2.7/0.6/3.3 | ⅓− | 82 | 84 | 60 | 63 | 79 | 81 |
| | | ⅕− | 81 | 81 | 68 | 72 | 10 | 13 |
| | | 1/10− | 21 | 24 | 64 | 67 | 10 | 13 |
| | 0.78/0.34/1.1 | ⅓− | 85 | 90 | 30 | 35 | 14 | 19 |
| | Effects of the | ⅓− | 0 | 0 | 4 | 6 | 4 | 7 |
| | substrates without | ⅓− | 0 | 0 | 0 | 0 | 0 | 0 |
| | presence of enzyme | | | | | | | |

A second test series was carried out under the same conditions with other microorganisms responsible for respiratory diseases in humans and which were detected in the cases of muscoviscidosis: tobramycin-resistant Burkholderia cepacia (ATCC BAA-245), mucoid Pseudomonas aeruginosa, Staphylococcus aureus resistant to methicillin and to oxacillin (ATCC 43300).

TABLE 9

| | | | HUMANS | | |
|---|---|---|---|---|---|
| | | | | Pseudomonas aeruginosa | Staphylococcus aureus (MRSA) |
| | | | Burkholderia cepacia | (mucoid) | methylcillin and oxacillin resistant |
| | | | Contact time | Contact time | Contact time |
| LPO | Before enzymatic reaction | | 5 minutes | 5 minutes | 5 minutes |
| U/mL | KI/KSCN/H202 | Dilution | % inhibition | % inhibition | % inhibition |
| 50 | 5.4/1.2/6.6 | 1/1− | 100 | 100 | 100 |
| | | ⅓− | 100 | 100 | 100 |
| | | ⅕− | 78 | 57 | 56 |
| | | 1/10− | 32 | 38 | 36 |
| | Effects of the substrates | 1/1− | 0 | 0 | 0 |
| | without presence of enzyme | ⅓− | 0 | 0 | 0 |

In summary, a strong antimicroorganism activity was detected.

### Example 13: Activity of a Composition According to the Invention Against the Microorganisms Responsible for the Deterioration of Plants, Fruits and Vegetables and Other Harvested Plants, in Particular in Bananas

A composition according to the invention was prepared by bringing together of potassium iodide (KI) 5.4 mM, 1.2 mM of potassium thiocyanate (KSCN), 6.6 mM of hydrogen peroxide (H₂O₂) in an ammonium acetate buffer 100 mM, pH 4.5 in the presence of 50 mg/L of lactoperoxidase (LP) (1000 ABTS units per mg) according to the protocol described in Example 1.

This composition was tested on contaminated bananas, in particular on bananas contaminated by fungi causing anthracnose lesions and crown rot.

By dipping the bananas in the composition prepared according to the protocol described in Example 1, it was demonstrated that said composition had a great efficacy against the infections caused by Colletotricum musae, Fusarium monoliforme and Fusarium oxysporum

In addition, it was demonstrated that the composition prepared according to Example 1 was more active against fungi compared to conventional fungicides which are toxic and pollute the environment.

### Example 14: Activity on Xylella fastidiosa

A composition according to the invention was prepared by bringing together of potassium iodide (KI) 5.4 mM, 1.2 mM of potassium thiocyanate (KSCN), 6.6 mM of hydrogen peroxide (H₂O₂) in a citrate-phosphate buffer 100 Mm, pH 6.9 in the presence of 50 mg/L of lactoperoxidase (LP) (1000 ABTS units per mg) according to the protocol described in Example 1.

The composition is tested for its bactericidal effect on Xylella fastidiosa subsp. fastidiosa, Xylella fastidiosa subsp. multiplex and Xylella fastidiosa subsp. pauca according to the protocol described below.

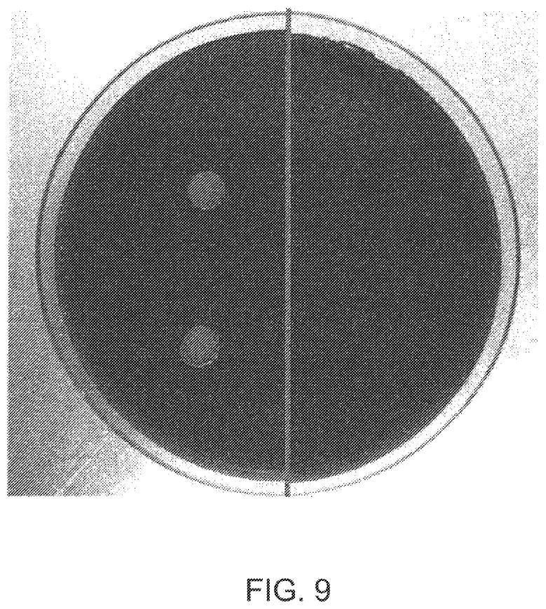

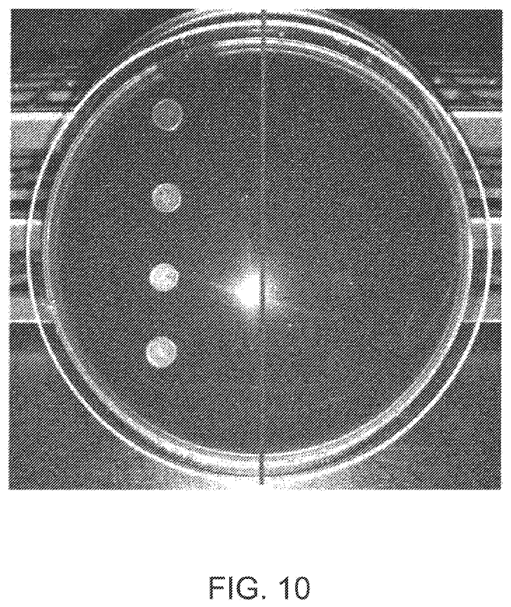

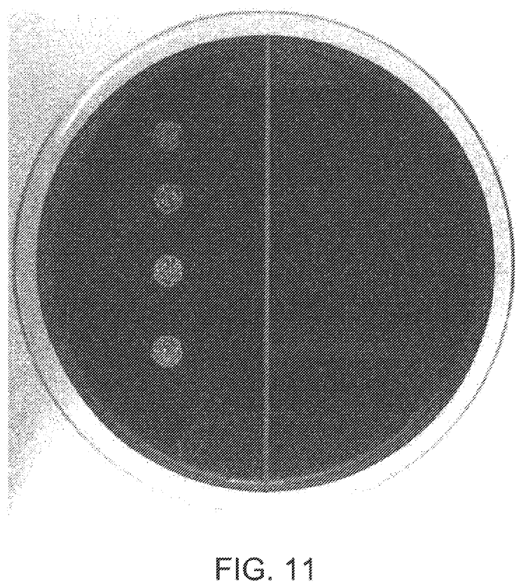

The results are presented in FIGS. 9, 10 and 11: the composition according to the invention has a bactericidal effect simultaneously on Xylella fastidiosa subsp. fastidiosa, Xylella fastidiosa subsp. multiplex and Xylella fastidiosa subsp. pauca.

The inoculum is prepared by 2 successive subcultures (each subculture was carried out at 26° C. for 10 days) on a medium comprising a mixture of 3 solutions A, B and C described below; the mixture of A and B being sterilized in the autoclave before addition of the solution C sterilized by filtration.
Solution A: 500 mL of distilled water (Aq. Dest) (50° C.)+10 g ACES Sigma A9758-25G
Solution B: 440 mL of distilled water (Aq. Dest)+40 mL 1.0 N KOH (1N KOH: 2.24 g solution 40 mL of distilled water.)

To solution B, 2 g of charcoal (Active Charcoal Sigma C-4386), 10 g of Yeast extract oxoid and 17 g of Agar are then added.

The mixture of the two solutions is prepared, followed by sterilization in the autoclave.
Solution C: cysteine HCl 0.4 g+ferric pyrophosphate 0.25 g solution 20 mL of distilled water (Aq. Dest) Cold sterilization (0.2 μm filter)

5 mL of sterile PBS are then added to a Petri dish comprising a Xylella strain; the Petri dish is plated with a sterile spatula, and the 5 mL are pipetted into a sterile flask.

The DO650 is adjusted to 1 (DO650 of 1=104 CFU/mL ref. Shi et al., 2007, Appl. Environ. Microbiol., 73 (21)) with sterile PBS.
in a 15-mL Falcon tube, add:

Control: 1 mL inoculum, 1 mL isotonic H₂O (NaCl 8.5 g/L) adjusted to pH 6.9 sterile, 1 mL of PD2 Broth

The PD2 broth is obtained by mixing the solutions A and B described below:

### Solution A:

Distilled water 1 L:
Soy peptone: 2.0 g
Bacto tryptone: 4.0 g
Disodium succinate: 1.0 g
Trisodium citrate: 1.0 g
K₂HPO₄: 1.5 g
KH₂PO₄: 1.0 g
Hemin chloride stock solution (0.1% in (0.05 N NaOH: 0.112 g/40 mL)): 10.0 mL
MgSO₄.7H₂O: 1.0 g
pH: 6.9

### Autoclave at 121° C. for 15 min.

### Solution B

Bovine serum albumin fraction V (20% w/v): 10 mL. Cold sterilize (0.2 μm filtration).

Mix solution A with solution B: when the autoclaved medium (A) has cooled to 50° C., add the sterilized albumin (B).

The biocontrol agent is obtained by mixing 1 mL of inoculum, 1 mL of inhibiting agent and 1 mL of PD2 broth, incubated under stirring (100 rpm) at 26° C. for 30 minutes.

The controls are obtained by mixing 4 drops (10 μL)/Petri dish; left side of the dish)×5 Petri dishes and incubation for 14 days 26° C.

The biocontrol agent by mixing 4 drops (10 μL)/Petri dish; to right side of the Petri dish)×5 Petri dishes and incubation for 14 days 26° C.

The Petri dishes are observed under the binocular microscope 30× and photographed.

The photographs are presented in FIGS. 9 to 11

### Example 15: Importance of the Removal of the Enzyme

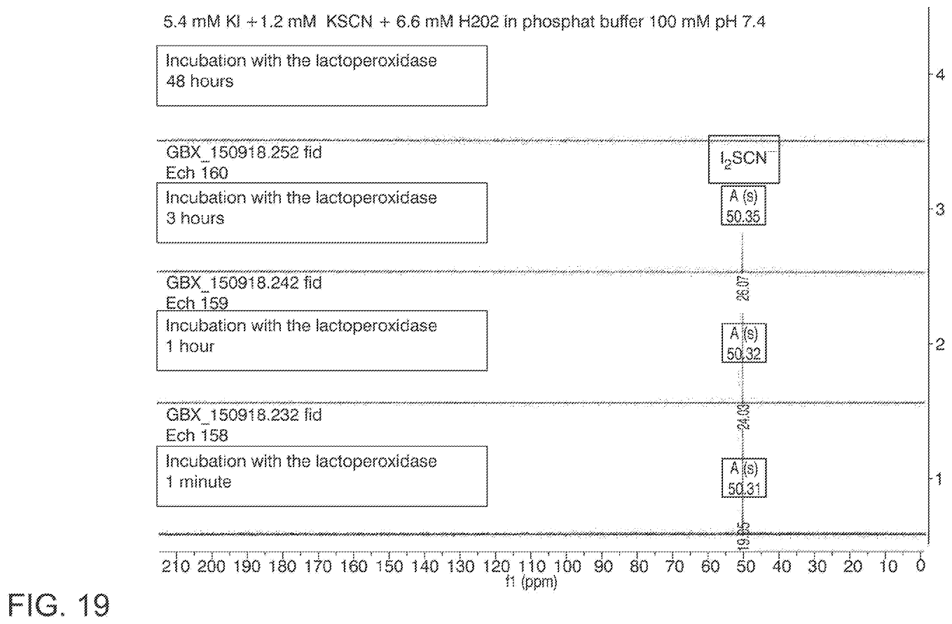

During the production of the wanted ions, if one keeps the enzyme in the mixture, one observes a gradual loss of the wanted ions, and, after 48 h, a total loss of the wanted ions due to enzymatic oxidation of the wanted ions, as illustrated in FIG. 19 in which one observes a decrease in the intensity of the signal as a function of the time of presence of the enzyme in the matrix (solution).

### Example 16: Addition of Supplementary Enzymes

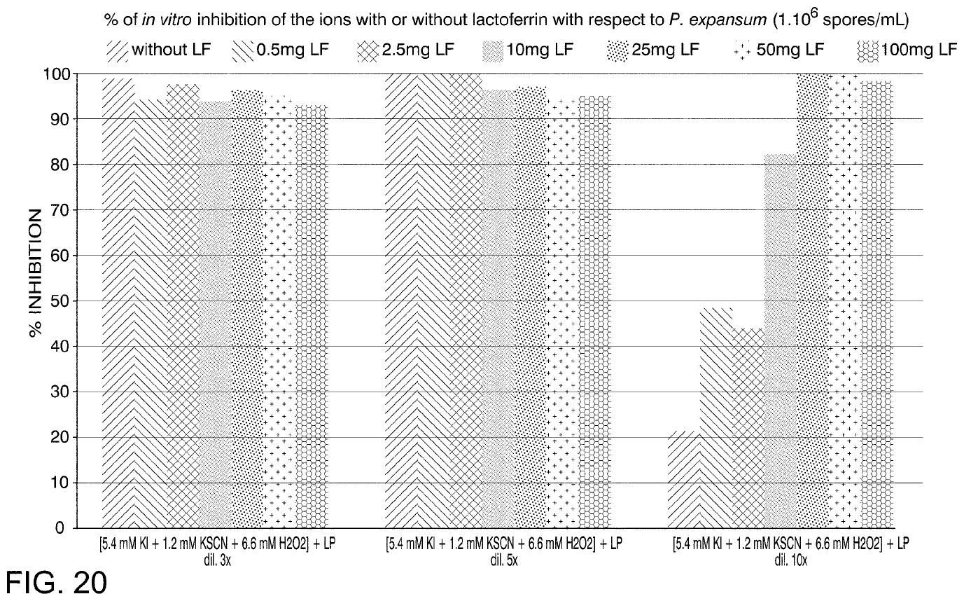

The presence of supplementary enzymes confers an unexpected effect, as illustrated in FIG. 20.

One observes notably that the addition of lactoferrin (>5 mg/L) confers an improved antimicrobial activity compared to the solutions without addition of lactoferrin, when the mixture is diluted 10-fold.

### Example 17: Comparison with the Prior Art

Non-obtention of the species I₂SCN⁻ or I(SCN)₂⁻.
Compositions comprising an enzymatic mixture with a KI/KSCN ratio of 1.74 were prepared according to the protocol described in EP1349457.

Compositions comprising an enzymatic mixture with a KI/KSCN ratio of 1.55 were prepared according to the protocols described in WO00/01237.

The NMR spectra were prepared under the following conditions: a Bruker AMX-500 MHz apparatus with an 8-mm broadband probe was used. The spectra were obtained from the reaction mixture (lactoperoxidase/[¹³C] SCN⁻/I⁻/H₂O₂ according to the described method. The sample consists of 540 μL of reaction mixture, 60 μL D₂O (deuterium oxide), 2 μL DSS (4,4-dimethyl-4-silapentane-1-sulfonic acid). The samples were placed in an NMR tube having a length of 8 mm and an 8-inch wall. The spectra were collected using the following parameters: scanning width=15 009, number of points=32000, acquisition time=1.066 s, recycling delay of 2 s, number of scans=2000. The chemical shifts (ppm) were referenced with respect to the NMR spectroscopy calibration standard, DSS (4,4-dimethyl-4-silapentane-1-sulfonic acid).

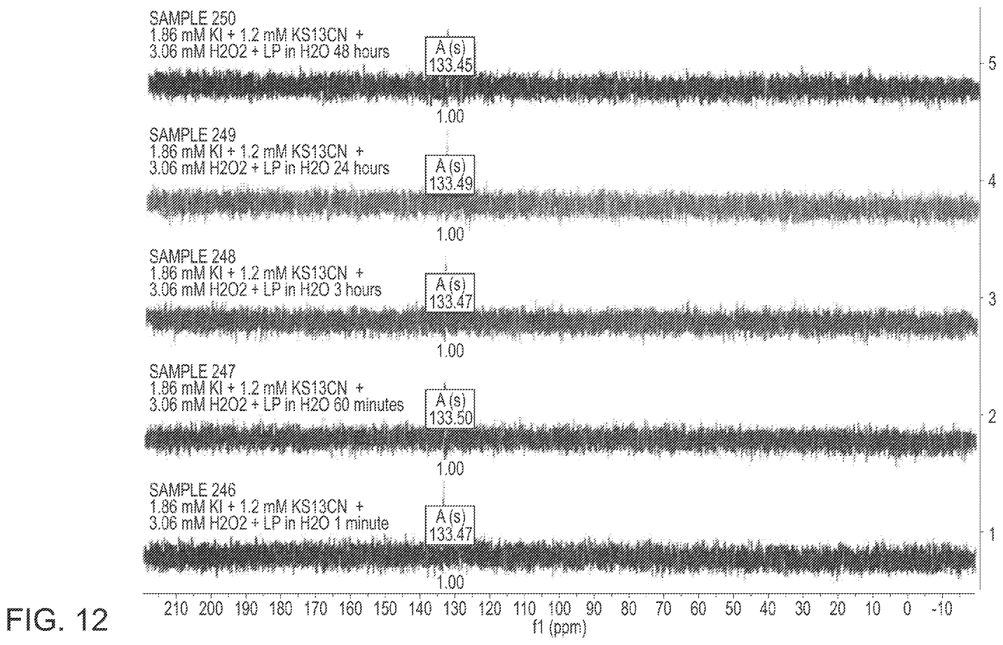

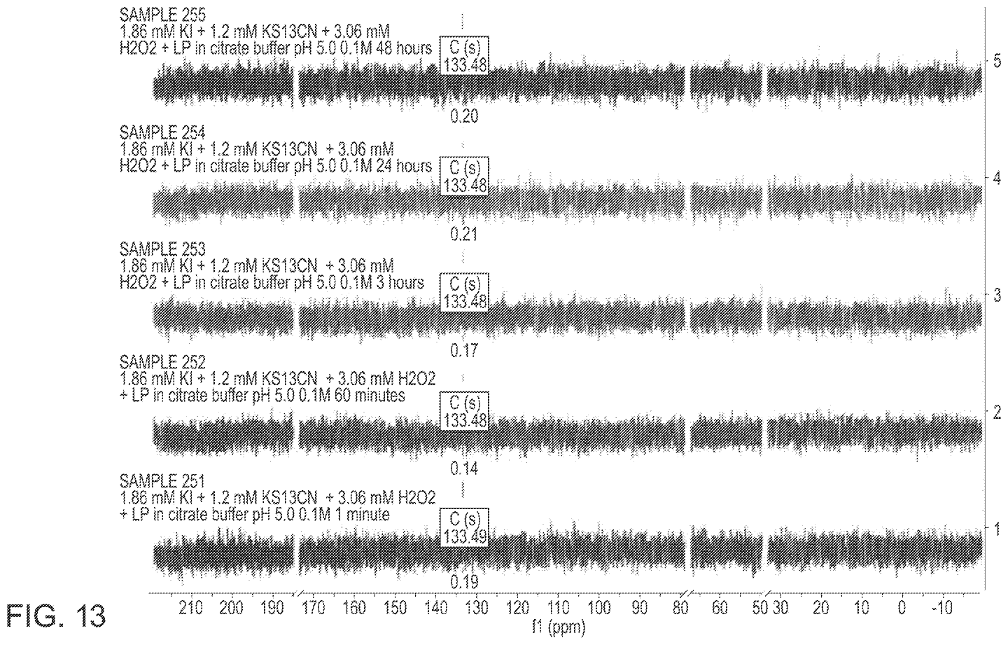

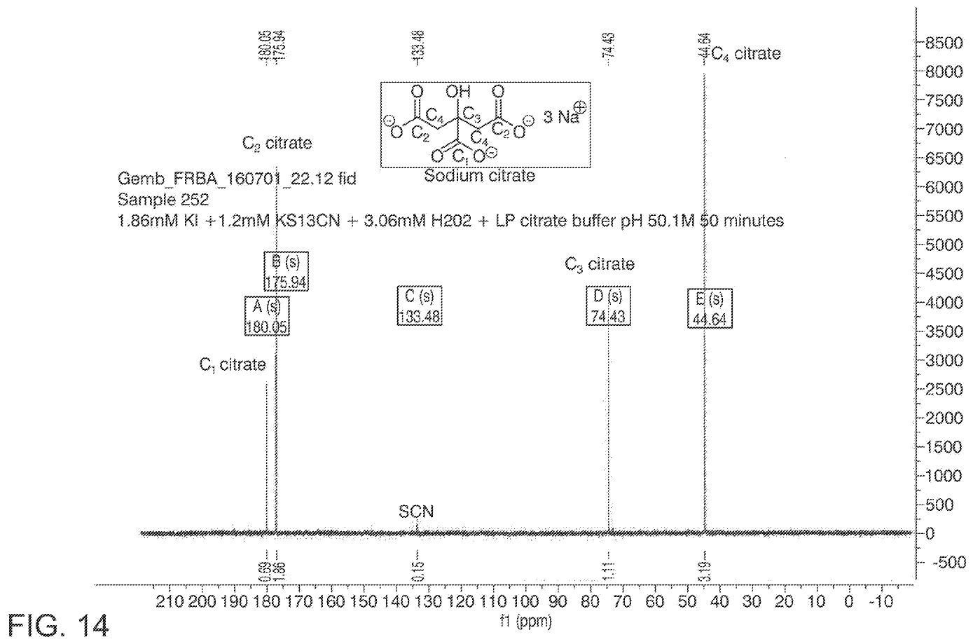

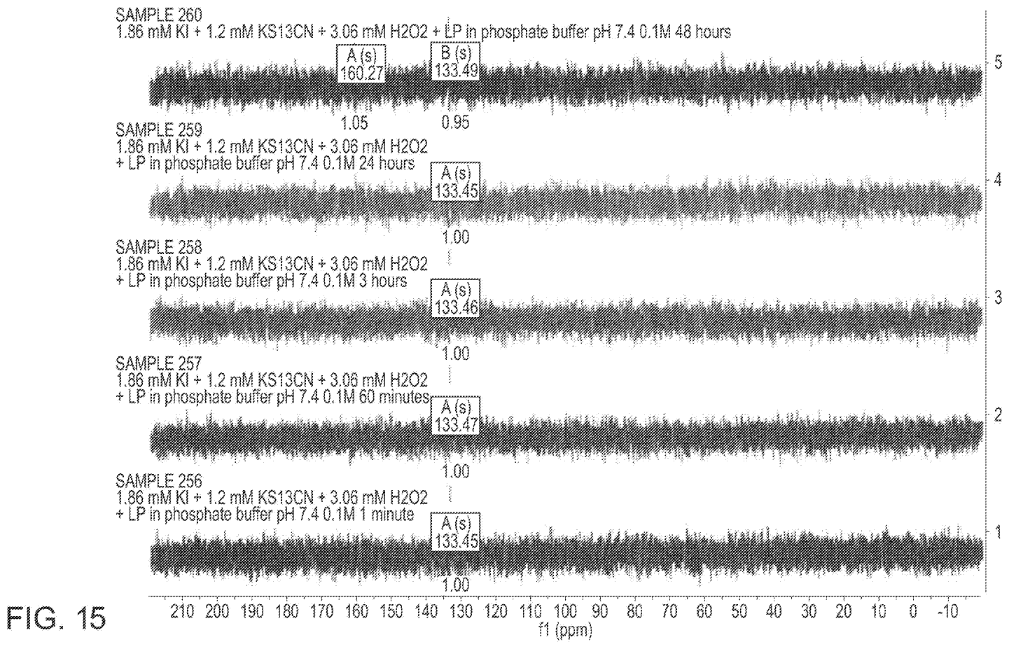

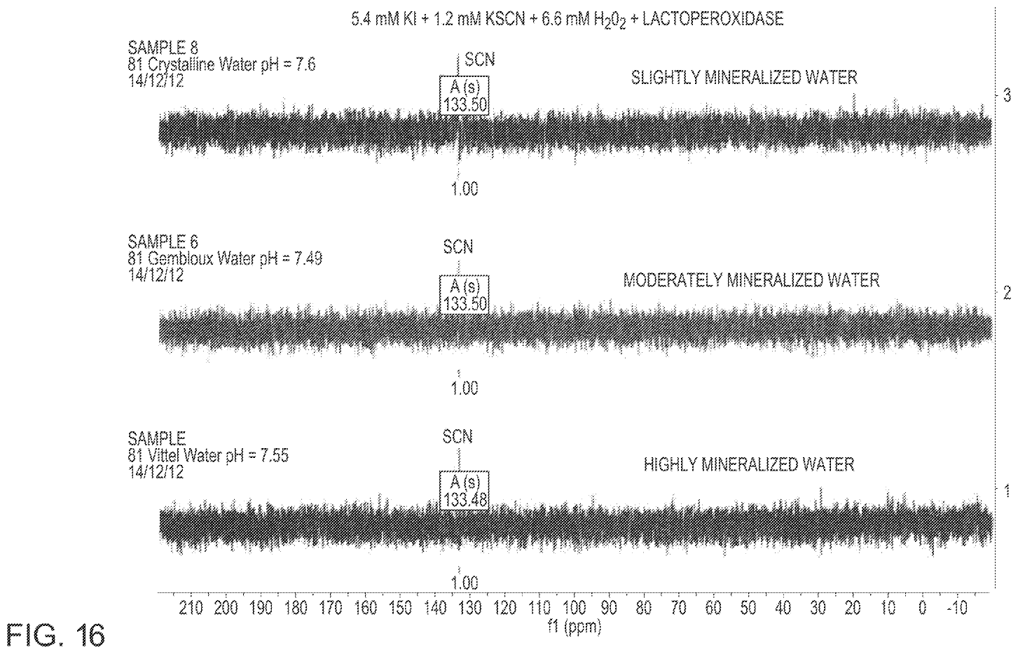

FIGS. 12 to 16 are the spectra obtained with the following compositions:
FIG. 12: Mixture of 1.86 mM KI+1.2 mM KSCN (ratio 1.55)+3.06 mM H₂O₂ in an aqueous matrix in the presence of lactoperoxidase. Removal of the enzyme after 1 minute, 60 minutes, 3 hours, 24 hours or 48 hours.
FIG. 13: Mixture of 1.86 mM KI+1.2 mM KSCN (ratio 1.55)+3.06 mM H₂O₂ in an acid-buffered matrix (citrate buffer 100 mM pH 5) in the presence of lactoperoxidase for 1 minute, 60 minutes, 3 hours, 24 hours or 48 hours. The signals corresponding to the citrate buffer were eliminated for greater clarity.
FIG. 14: Illustration of the signals corresponding to the citrate buffer and to the SCN⁻.
FIG. 15: Mixture of 1.86 mM KI+1.2 mM KSCN (ratio 1.55)+3.06 mM H₂O₂ in a neutral buffered matrix (phosphate buffer 100 mM pH 7.4) in the presence of lactoperoxidase for 1 minute, 60 minutes, 3 hours, 24 hours or 48 hours.
FIG. 16: Mixture 5.4 mM KI+1.2 Mm KSCN+6.6 Mm H₂O₂ in the presence of lactoperoxidase.

The peak corresponding to KS¹³CN is observed regardless of which matrix is used. There are no peaks at 49-50 ppm. In the mixtures described in WO00/01237 or EP1349457, there is no production of I₂SCN⁻ or I(SCN)₂⁻ ion.

### Demonstration of the Presence of Hypothiocyanite Ions.

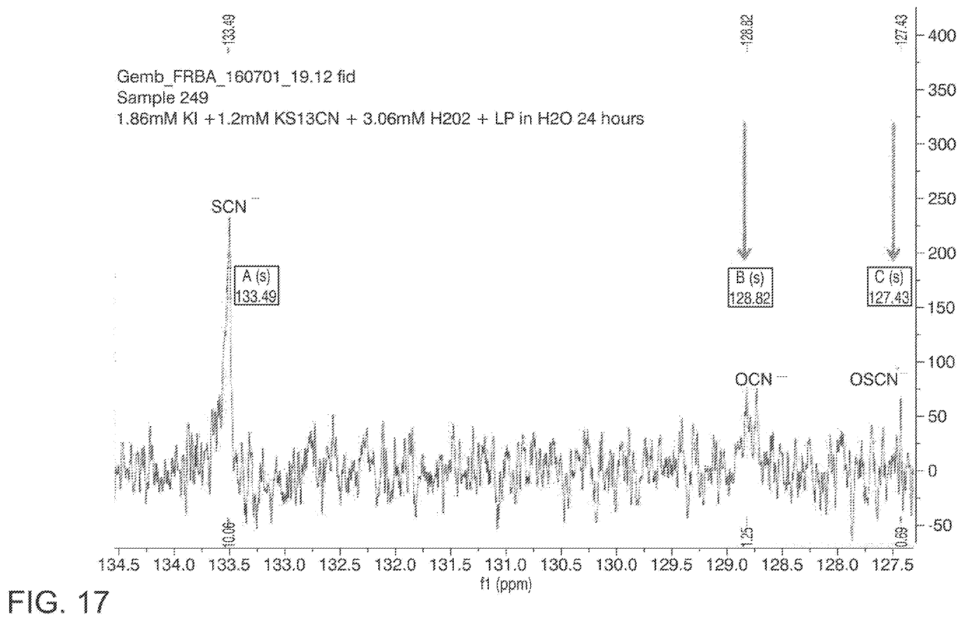

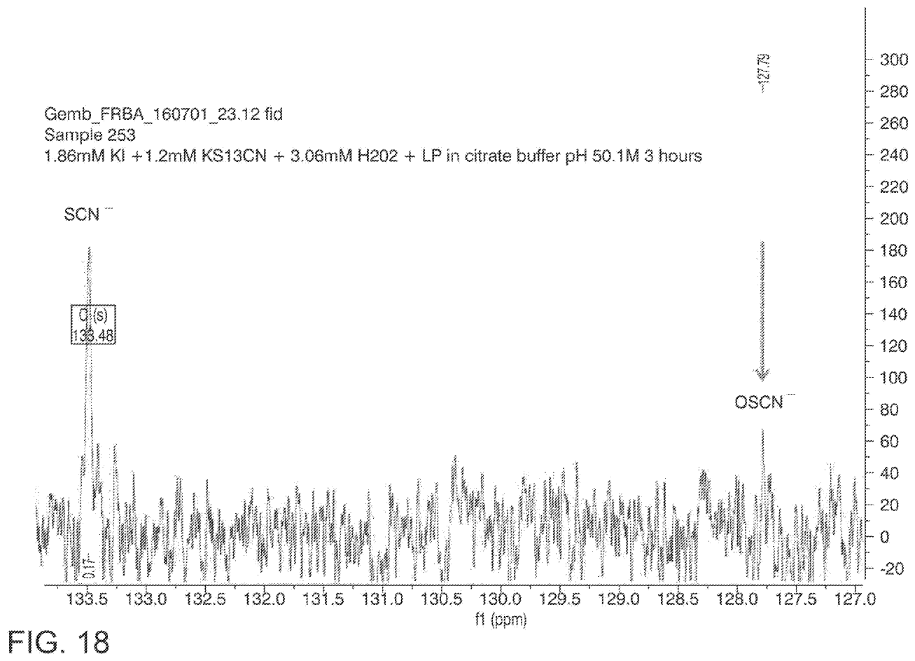

FIG. 17: Mixture of 1.86 mM KI+1.2 mM KSCN (ratio 1.55)+3.06 mM H₂O₂ in an aqueous matrix in the presence of lactoperoxidase. Removal of the enzyme after 24 hours.
FIG. 18: Mixture of 1.86 mM KI+1.2 mM KSCN (ratio 1.55)+3.06 mM H₂O₂ in an acid buffered matrix (citrate buffer 100 mM pH 5) in the presence of lactoperoxidase for 3 hours.

In the enzymatic mixtures prepared according to WO00/01237 or EP1349457, the hypothiocyanite ions are detected in small quantity as are the cyanate ions (OCN⁻), perfectly identifiable thanks to its triplet signal (Gerritsen et al. 1993), which correspond to the degradation of the OSCN⁻ ions.

### Example 18: Comparison of the Kinetics of the Enzymatic and Chemical Oxidations and the Antimicrobial Activities

A) Rapidity of the production of the wanted ions (enzymatic kinetics) which implies immediate antimicrobial activity for the enzymatic mixture.

Kinetics of production of the wanted ions (measurement by oxidation of the —SH or —NH₂ groups)

Solutions comprising 5.4 mM KI+1.2 mM KSCN+6.6 mM H₂O₂ in a sodium acetate buffer 100 mM pH 4.4+/−LP according to the invention described are prepared

TABLE 10

| | |
|---|---|
| | With Lactoperoxidase: incubation |
| | for 1 minute then removal of the |
| Without Lactoperoxidase | lactoperoxidase |

| Time | Oxidation -SH | Time | Oxidation -SH |
|---|---|---|---|
| (min) | (μM/L) | (min) | (μM/L) |
| 0 | 4 | 0 | 517 |
| 3 | 49 | 3 | 568 |
| 5 | 74 | 5 | 586 |
| 10 | 133 | 10 | 587 |
| 15 | 168 | 15 | 587 |
| 20 | 183 | 20 | 585 |
| 30 | 197 | 30 | 582 |
| 60 | 203 | 60 | 702 |
| 120 | 235 | 120 | 705 |

TABLE 11

| | |
|---|---|
| | With Lactoperoxidase: incubation |
| | for 1 minute then removal of the |
| Without Lactoperoxidase | lactoperoxidase |

| | Oxidation | | Oxidation |
|---|---|---|---|
| Time | -NH₂ | Time | -NH₂ |
| (min) | (μM/L) | (min) | (μM/L) |
| 1 | 53 | 1 | 2521 |
| 3 | 54 | 3 | 1924 |
| 5 | 107 | 5 | 1426 |
| 10 | 158 | 10 | 1194 |
| 15 | 218 | 15 | 991 |
| 20 | 220 | 20 | 970 |
| 30 | 253 | 30 | 829 |
| 60 | 308 | 60 | 947 |

One notes that the kinetics of formation of the wanted ions is entirely different: the production of the wanted ions with the enzyme is instantaneous, while the production obtained without the enzyme is rather slow, after 1 hour of incubation only approximately ⅓ of the wanted ions Is obtained. This has an implication for the immediate activity of the active mixture:

Activity with Respect to Penicillium expansum

TABLE 12

| in vitro inhibition with respect to 1 10⁶ spores/mL of | | |
|---|---|---|
| Penicillium expansum of the mixture (5.4 mM KI + 1.2 | | |
| mM KSCN + 6.6 mM H₂O₂) without Lactoperoxidase | % inhibition | % inhibition |
| (chemical mixture) | after 48 h | after 120 h |
| 5.4 mM KI + 1.2 mM KSCN + 6.6 mM H₂O₂ tap water ⅓ | 34 | 4 |
| 5.4 mM KI + 1.2 mM KSCN + 6.6 mM H₂O₂ tap water ⅕ | 30 | 33 |
| 5.4 mM KI + 1.2 mM KSCN + 6.6 mM H₂O₂ tap water 1/10 | 25 | 32 |
| 5.4 mM KI + 1.2 mM KSCN + 6.6 mM H₂O₂ tap water 1/15 | 25 | 7 |
| 5.4 mM KI + 1.2 mM KSCN + 6.6 mM H₂O₂ tap water 1/20 | 18 | 3 |
| 5.4 mM KI + 1.2 mM KSCN + 6.6 mM H₂O₂ tap water 1/30 | 18 | 29 |
| 5.4 mM KI + 1.2 mM KSCN + 6.6 mM H₂O₂ + tap water pH 4.4 ⅓ | 17 | 0 |
| 5.4 mM KI + 1.2 mM KSCN + 6.6 mM H₂O₂ + tap water pH 4.4 ⅕ | 9 | 0 |
| 5.4 mM KI + 1.2 mM KSCN + 6.6 mM H₂O₂ + tap water pH 4.4 1/10 | 14 | 2 |
| 5.4 mM KI + 1.2 mM KSCN + 6.6 mM H₂O₂ + tap water pH 4.4 1/15 | 16 | 27 |
| 5.4 mM KI + 1.2 mM KSCN + 6.6 mM H₂O₂ + tap water pH 4.4 1/20 | 14 | 22 |
| 5.4 mM KI + 1.2 mM KSCN + 6.6 mM H₂O₂ + tap water pH 4.4 1/30 | 15 | 23 |
| 5.4 mM KI + 1.2 mM KSCN + 6.6 mM H2O2 + acetate buffer 0.1M pH 4.5 ⅓ | 16 | 21 |
| 5.4 mM KI + 1.2 mM KSCN + 6.6 mM H₂O₂ + acetate buffer 0.1M pH 4.5 ⅕ | 31 | 17 |
| 5.4 mM KI + 1.2 mM KSCN + 6.6 mM H₂O₂ + acetate buffer 0.1M pH 4.5 1/10 | 21 | 21 |
| 5.4 mM KI + 1.2 mM KSCN + 6.6 mM H₂O₂ + acetate buffer 0.1M pH 4.5 1/15 | 26 | 37 |
| 5.4 mM KI + 1.2 mM KSCN + 6.6 mM H₂O₂ + acetate buffer 0.1M pH 4.5 1/20 | 29 | 42 |
| 5.4 mM KI + 1.2 mM KSCN + 6.6 mM H₂O₂ + acetate buffer 0.1M pH 4.5 1/30 | 35 | 35 |

TABLE 13

| In vitro inhibition with respect to 1 10⁶ | | |
|---|---|---|
| spores/mL of Penicillium expansum of the | | |
| mixture (5.4 mM KI + 1.2 mM KSCN + 6.6 | | |
| mM H₂O₂) with Lactoperoxidase | % inhibition | % inhibition |
| for 1 minute (enzymatic mixture) | after 48 h | after 120 h |
| B1 Acetate Buffer 0.5M pH 4.5 ⅓ | 96 | 97 |
| B1 Acetate Buffer 0.5M pH 4.5 ⅕ | 96 | 97 |
| B1 Acetate Buffer 0.5M pH 4.5 1/10 | 96 | 98 |
| B1 Acetate Buffer 0.5M pH 4.5 1/15 | 96 | 98 |
| B1 Acetate Buffer 0.5M pH 4.5 1/20 | 97 | 98 |
| B1 Acetate Buffer 0.5M pH 4.5 1/30 | 95 | 60 |
| B1 Acetate Buffer 0.5M pH 4.5 1/50 | 81 | 37 |
| B1 Acetate Buffer 0.1M pH 4.5 ⅓ | 94 | 96 |
| B1 Acetate Buffer 0.1M pH 4.5 ⅕ | 96 | 97 |
| B1 Acetate Buffer 0.1M pH 4.5 1/10 | 95 | 97 |
| B1 Acetate Buffer 0.1M pH 4.5 1/15 | 97 | 98 |
| B1 Acetate Buffer 0.1M pH 4.5 1/20 | 70 | 36 |
| B1 Acetate Buffer 0.1M pH 4.5 1/30 | 53 | 22 |
| B1 Acetate Buffer 0.1M pH 4.5 1/50 | 40 | 4 |
| B1 Acetate Buffer 0.01M pH 4.5 ⅓ | 93 | 96 |
| B1 Acetate Buffer 0.01M pH 4.5 ⅕ | 94 | 96 |
| B1 Acetate Buffer 0.01M pH 4.5 1/10 | 94 | 97 |
| B1 Acetate Buffer 0.01M pH 4.5 1/15 | 81 | 99 |
| B1 Acetate Buffer 0.01M pH 4.5 1/20 | 53 | 0 |
| B1 Acetate Buffer 0.01M pH 4.5 1/30 | 19 | 0 |
| B1 Acetate Buffer 0.01M pH 4.5 1/50 | 6 | 0 |
| B1 Acetate Buffer 0.001M pH 4.5 ⅓ | 100 | 100 |
| B1 Acetate Buffer 0.001M pH 4.5 ⅕ | 97 | 99 |
| B1 Acetate Buffer 0.001M pH 4.5 1/10 | 96 | 99 |
| B1 Acetate Buffer 0.001M pH 4.5 1/15 | 95 | 99 |
| B1 Acetate Buffer 0.001M pH 4.5 1/20 | 29 | 0 |
| B1 Acetate Buffer 0.001M pH 4.5 1/30 | 8 | 0 |
| B1 Acetate Buffer 0.001M pH 4.5 1/50 | 13 | 0 |

One notes that the chemical mixture (incubation time of the reagents: 1 minute) is not effective for in vitro growth inhibition of Penicillium expansum.

On the contrary, the enzymatic mixture of (5.4 mM KI+1.2 mM KSCN+6.6 mM 1H₂O₂)+lactoperoxidase for 1 minute, then removal of the enzyme, is effective up to the 1/30 dilution for the buffer 500 mM, up to 1/15 dilution with the buffer 100 mM, 10 mM and 1 mM:

The method for preparing the active mixture containing the wanted ions, that is to say chemical or enzymatic, has an implication for the immediate antimicrobial activity of the mixture. The enzymatic mixture has an immediate antimicrobial efficacy (present as early as after 1 minute of incubation), while this antimicrobial activity is absent after 1 minute of incubation of the substrates in the chemical mixture.

### Example 19: Antimicrobial Activity with Respect to E. Col of a Composition Immobilized on a Fabric and Lyophilized

A composition according to the invention was prepared by bringing together of 5.4 mM of potassium iodide (KI), 1.2 mM of potassium thiocyanate (KSCN), 6.6 mM of hydrogen peroxide (H₂O₂) in the presence of 50 mg/L of lactoperoxidase (LP) (1000 ABTS units per mg) in a sodium citrate buffer 100 mM pH 6.2 (FIG. 23B) or a phosphate buffer 100 mM pH 7.4 (FIG. 23C) or a sodium citrate buffer 100 mM pH 6.2 and lyophilized after preparation and reconstituted in water (FIG. 23D). These compositions were immobilized on a fabric, and the antibacterial activity of these fabrics impregnated with these compositions was tested with respect to E. coli (FIGS. 23B, 23C and 23D).

It can be seen that the lyophilized composition maintains a bactericidal action equivalent to the other non-lyophilized compositions (FIGS. 23B and 23C versus FIG. 23D). The “control” fabric was impregnated with sterile water (FIG. 23A).

FIGS. 23A-D illustrate the action which the composition according to the invention has on E. coli (109 CFU/mL).
It is apparent that after 24 h of incubation at 37° C. of 100 μL of E. coli at 109 CFU/mL on a culture medium in a Petri dish, the composition immobilized on a fabric stops the growth of the bacterium (halo visible).

1. A stable composition obtained by enzymatic oxidation of a halide thiocyanate mixture, comprising at least one ion selected from the group consisting of the I₂SCN— ions and the I(SCN)₂— ions, said composition being free of hypothiocyanite ions (OSCN—).

2. The stable composition according to claim 1, further comprising iodine thiocyanate ISCN.

3. The stable composition according to claim 1, further comprising at least one compound selected from the group consisting of lactoferrin, lysozyme, immunoglobulins, and growth factors.

4. The stable composition according to claim 1, further comprising at least one compound selected from the group consisting of oils, spreading agents, emulsifiers, lubricants, and adhesives.

5. The stable composition according to claim 1, wherein the composition is configured to lose less than 20% of an antimicrobial activity in five months when the composition is stored in a closed container at 4° C. with protection from light.

6. The stable composition according to claim 1, consisting of at least one ion selected from the group consisting of the I₂SCN— ions and the I(SCN)₂— ions, and
optionally at least one selected from the group consisting of iodine thiocyanate, a lactoferrin, a lysozyme, an immunoglobulin, a growth factor, an oil, a spreading agents, an emulsifier, a lubricant, and an adhesive.

7. A method for manufacturing a stable composition according to claim 1, comprising:
a step A of preparation of a reaction medium comprising at least two substrates, at least one oxidizing agent, and a catalyst, the bringing together of said catalyst and said oxidizing agent being contingent upon the bringing together of said two substrates;
a reaction step B starting with the bringing together of said oxidizing agent and said catalyst;
a step C of removal of said catalyst, and of recovery of a composition according to the invention comprising at least one of the I₂SCN— ions and/or of the ions I(SCN)₂— ions;
said substrates being halide (X—) and thiocyanate (SCN—) ions,
said oxidizing agent being a hydrogen peroxide (H₂O₂) generating system and/or hydrogen peroxide, the catalyst being at least one peroxidase,
wherein the reaction step has a duration from 30 to 1800 seconds and in that it does not give rise to the formation of hypothiocyanite ion (OSCN—).

8. The method according to claim 7, further comprising a step of lyophilization of the composition at the end of which a lyophilisate is obtained, said lyophilisate enabling, during a redissolution, the reconstitution of said composition which includes at least one of the I₂SCN— ions and/or of the I(SCN)₂— ions and is free of hypothiocyanite ion (OSCN—).

9. The method according to claim 7, wherein the halide ion (X—) is selected from the group consisting of the iodide ion (I—), the bromide ion (Br—), and the chloride ion (Cl—).

10. The method according to claim 7, wherein the halide ion (X—) is the iodide ion (I—).

11. The method according to claim 7, wherein the ratio between the molar concentration of thiocyanate ion (SCN—) and the molar concentration of iodide ion (I—) is greater than 1.

12. The method according to claim 7, wherein the pH of the solution is from 4 to 8.

13. The method according to claim 7, wherein the contact time is from 30 to 200 seconds.

14. The method according to claim 7, wherein the peroxidase is selected from the group consisting of the lactoperoxidase (LP), the thyroid peroxidase (TPO), the myeloperoxidase (MPO), the salivary peroxidase (SPO) and the eosinophil peroxidase (EPO).

15. The method according to claim 7, wherein the peroxidase is the lactoperoxidase (LP).

16. The method according to claim 7, wherein said peroxidase has a concentration from 1 mg/L to 500 mg/L.

17. The method according to claim 7, wherein the thiocyanate ion (SCN—) is present at a molar concentration from 0.1 mM to 1 M.

18. The method according to claim 7, wherein the halide ion (X—) is the iodide ion (I—) and is present at a molar concentration from 0.1 mM to 1 M.

19. The method according to claim 18, wherein a I⁻/SCN⁻ ratio is in a range of from more than 1:1 to 9:1.
